(19) United States
(12) Patent Application Publication
(10) Pub. No.: US 2025/0160616 A1
(43) Pub. Date: May 22, 2025

(54) METHOD OF GENERATING A TRAINING DATA SET FOR DETERMINING PERIODONTAL STRUCTURES OF A PATIENT

(71) Applicant: 3SHAPE A/S, Copenhagen K (DK)

(72) Inventors: Stavroula MICHOU, Copenhagen K (DK); Mathias Schärfe LAMBACH, Copenhagen K (DK); Pia Elisabeth NØRRISGAARD, Copenhagen K (DK); Peter SØNDERGAARD, Copenhagen K (DK); Christoph VANNAHME, Copenhagen K (DK)

(73) Assignee: 3SHAPE A/S, Copenhagen K (DK)

(21) Appl. No.: 18/839,043

(22) PCT Filed: Feb. 15, 2023
(86) PCT No.: PCT/EP2023/053741

§ 371 Date: Aug. 16, 2024

(30) Foreign Application Priority Data
Feb. 18, 2022 (EP) 22157583.0

(51) Int. Cl. A61B 1/00; A61B 1/24; G06V 10/26; G06V 10/774

(52) U.S. Cl. CPC A61B 1/000096; A61B 1/00045; A61B 1/24; G06V 10/26; G06V 10/774; G06V 2201/03

(57) ABSTRACT

According to an embodiment, a dental scanning system and a computer implemented method of generating a training set for a machine learning model are disclosed. According to a further embodiment, a dental scanning system and a computer implemented method for providing a diagnostic data set of a patient are disclosed.

### FIELD

The disclosure relates to generating training data set for a machine learning model and for providing a diagnostic data set to a patient including periodontal properties. In particular, the disclosure relates to a method, a computer program, a data processing device, and a computer-readable storage medium for generating training data set to be used for diagnosing or at least aid in diagnosing one or more periodontal structures of a patient's mouth.

### BACKGROUND

The clinical methods for assessing bone level and correlated bone loss are today very time-consuming, unpleasant for the patient and expensive (Shayeb et al., 2014; Guo et al., 2016, Fiorellini et al., 2021). In particular, radiographs (intraoral/panoramic) or even a Cone Beam Computed Tomography (CBCT) scan are employed to investigate the bone level or assess the possible bone loss (Fiorellini et al., 2021). However, ionizing radiation dose should be avoided as it increases the cancer risk, and while CBCT scans offer the highest quality for the determination of the bone level, they also pose the highest radiation dose. Additionally, when for example assessing the bone level in relation to implant placement, more invasive methods like transgingival probing or surgical exploration are also considered the clinical gold standard (Kloukos et al., 2021).

Based on the above, there is a clear need to develop a clinically applicable, non-invasive and reproducible method that will be able to estimate the bone level and possible bone loss without the use of ionizing radiation.

### SUMMARY

Normally, assessment of alveolar bone, is performed by a dentist assessing patterns and extent of the alveolar bone (i.e. how the bone looks and where the bone is) using invasive methods or radiographs. Generally, the bone level is measured from the cementoenamel junction (CEJ) to the crest of alveolar bone, whereas bone loss is defining a process meaning that if the bone level is not normal some bone has been resorbed resulting in bone loss.

Generally, a bone loss is considered present when the bone level measured as a distance between the CEJ to the crest of the alveolar bone exceeds 3 mm. The regular method using radiographs to assess bone level should be avoided as it exposes the patient to unnecessary radiation dose. The methods described herein substantially aim at eliminating the use of radiation devices when assessing periodontal structures.

Accordingly, an aspect of the present disclosure is to obtain a fast and reliable method for determining one or more periodontal structures of a patient, and for aiding the diagnosis of diseases or dental conditions of a patient based on the one or more periodontal structures. The one or more periodontal structures may be bone loss and/or bone level.

A further aspect of the present disclosure is to obtain a method for determining one or more periodontal structures of a patient which is safer for the patient. i.e. minimizing the exposure of ionizing radiation as generally used in the current methods of assessing especially bone loss.

The present disclosure addresses the above-mentioned challenges, by providing a computer implemented method using a machine learning model to assess the bone level and the correlated bone loss in intra-oral surface scan images. According to the aspects, a computer implemented method for generating a training data set for a machine learning model is disclosed. The method comprising receiving extra-oral image data of a plurality of candidate patients provided by an extra-oral image device and determining one or more periodontal structures for each of plurality of candidate patients based on the received extra-oral image data.

The extra-oral image device may be an X-ray scanner configured to scan a patient based on ionized radiation. The extra-oral image device may be a cone beam computed tomography (CBCT) scanner configured to scan the patient's head, an X-ray image device, or an intra-oral radiograph (X-ray) configured to scan part of a patient teeth. The extra-oral image data may be provided by an image device, such as the ones previously defined extra-oral imaging device. Extra-oral image data is extra-oral scan data as output form the extra-oral image device.

Furthermore, the method comprising receiving intra-oral scan data of the plurality of candidate patients provided by an intra-oral scanner and generating a training data set by combining the extra-oral image data with the intra-oral scan data for each of the plurality of candidate patients for aligning the one or more periodontal structures to the intra-oral scan data.

In the context of this application combining is considered to comprise the process of image alignment, where elements in one image is mapped into meaningful correspondence with elements in a second image. In an example according to the disclosure, the determined one or more periodontal structures as determined from the extra-oral image data may be considered as elements or more specifically image labels that are to be correlated or mapped with elements in the intra-oral scan data. The elements of the intra-oral scan data may e.g. be teeth and/or gingiva.

Considering an example, the combining may comprise; identifying corresponding teeth in the extra-oral image data and the intra-oral scan data. Determining in the extra-oral image data one or more periodontal structures (i.e. labels, such as e.g. a bone level) and using an image alignment process to map or correlate the determined periodontal structures with corresponding teeth in the intra-oral scan data.

The image alignment process may result in the generation of e.g. a mapping matrix comprising information in the form of tooth number and correlated label information—i.e. a matrix representing the periodontal structures determined from the extra-oral image data together with the teeth at which a periodontal structure has been determined. This mapping matrix may be used together with the intra-oral scan data to form the training data set. That is, for each intra-oral scan data at least one corresponding mapping matrix with extra-oral periodontal structure (i.e. label) information is provided. In this way it is ensured that the periodontal structures determined from the extra-oral image data may be correlated in the form of image alignment with the intra-oral scan data.

In another example, the combining may comprise; identifying corresponding teeth in the extra-oral image data and the intra-oral scan data. Determining in the extra-oral image data one or more periodontal structures (i.e. labels, such as e.g. a bone level) and using an image alignment process to map or correlate the determined periodontal structures with corresponding teeth in the intra-oral image data. In this example the alignment process may comprise the step of image registration, where the periodontal structures determined from the extra-oral image data is transferred to the intra-oral scan data in an image warping procedure. In both of the mentioned examples, a transformation (i.e. mapping) matrix, ensuring that the teeth numbering and corresponding labels of the extra-oral image data is correlated with teeth of the intra-oral scan data and that the determined periodontal structures for each teeth of the extra-oral image data are correlated with the intra-oral data, is generated.

With these methods of combining, it is possible to use the intra-oral scan data as input to the machine learning model to train it, where the periodontal structures determined from the extra-oral image data may be considered to constitute the labels of the training data set (i.e. the labels could be considered the ground truth of the data). The mapping matrices for each of the candidate patients extra-oral and intra-oral image data is generated to ensure that the output of the machine learning model (as generated from the intra-oral scan data input) can be compared to the ground truth as extracted from the extra-oral image data during a training phase of the machine learning model.

With the combination of extra-oral and intra-oral scan data to generate the training data set it is ensured that the extra-oral information of e.g. bone level, which is not be possible to determine from the intra-oral data, is correlated with the intra-oral scan data to generate the training dataset. Training a machine learning model based on a training data set having extra-oral image information that can be used together with intra-oral scan data information ensures that the machine learning model upon training is able to learn how to extract periodontal feature information from intra-oral scan data without having the extra-oral image data as source of information. Normally periodontal structures would not be identifiable from intra-oral scans alone, but when training a machine learning model on the two different datasets (i.e. the extra-oral image data and the intra-oral image data) it is possible to create a machine learning model which is capable of identifying such periodontal structures when being presented with only intra-oral surface scan information.

Accordingly, for the training of the machine learning model the method comprises that for each intra-oral scan data acquired a corresponding extra-oral image of the same teeth taken at substantially the same time is acquired. In this way it is ensured that the determined periodontal structures for each of the extra-oral image data can be combined with the respective intra-oral scan data. The training data set thus comprises a plurality of extra-oral image data and intra-oral scan data, where a preprocessing step comprises determining in the extra-oral image data one or more periodontal structures and subsequently generating a mapping matrix comprising the determined periodontal structure and related determined periodontal structure for each of the teeth in the extra-oral image data.

In an example the mapping matrix may comprise further information, such as patient information, e.g. age, gender, diseases etc. as identified from a previous obtained extra-oral image data and/or intra-oral scan data.

Further, the mapping matrix may comprise information about location of the determined periodontal structure, such as if the periodontal structure has been identified on the buccal or lingual side of the tooth, at what given reference site the periodontal structure was identified etc.

The intra-oral scanner described herein may be a scanner configured to scan the anatomy of the mouth of a patient without the use of ionizing radiation. The intra-oral scanner may be configured to scan the mouth of the patient by arranging at least a scanner tip of the intra-oral scanner within the mouth of the patient and moving the scanner tip around and inside the mouth during the scanning. The intra-oral scanner may be a 2D intra-oral camera or a 3D intra-oral scanner. Preferably, the intraoral scanner is a handheld intra-oral scanner which can be applied in a clinical practice and controlled by hand by a dental practitioner when scanning a patient during a clinical visit.

As already described, the aspect described herein solves the problem of eliminating the use of ionizing radiation when e.g. assessing periodontal diseases and change in periodontal structures of a patient. Instead of using extra-oral image data exposing the patient to radiation, the trained machine learning model is capable of identifying periodontal structures in intra-oral scan data which before would only been possible to identify on e.g. CBCT data, X-ray data or similar, thus eliminating the patient exposure to radiation.

Accordingly, the general idea described is to use the extra-oral image data in generating the training dataset to identify periodontal structures, such as bone level and related bone loss, and subsequently form a mapping transformation (i.e. the mapping matrix) that transforms the extra-oral image data (i.e. the periodontal feature labels) to e.g. annotations of the intra-oral scan data of the training dataset. In this way a training dataset consisting of intra-oral scans with labels (also to be considered as annotations) generated from an extra-oral data source is generated. This allows using intra-oral scan data with periodontal structure information, which is normally not obtainable in intra-oral scan data, to be input to a machine learning model for training such model.

By using the described training setup of combining data from an extra-oral image and an intra-oral scan, it is possible to output from the trained machine learning model, a diagnostic dataset which comprises information about periodontal structures directly obtained from an intra-oral surface scan, without the use of previously obtained extra-oral image data information of that scan. This eliminates the risks associated with radiograph imaging as the radiation images taken by an extra-oral imaging device is no longer needed for assessing periodontal structures in a patient.

The term “candidate patient” in the context of the disclosure, is a patient which is being scanned by an extra-oral image device and an intra-oral scanner during a visit at a dental clinic. Both scans may not be performed during the same visit, but it may be preferred that both scans are taken within a short time period to avoid large changes in the periodontal structures, e.g. that the bone level between acquiring the extra-oral image data and the intra-oral image data has changed a lot. To ensure accurate training of the machine learning model, it is important that the periodontal structure information determined from the extra-oral data is assessed at substantially the same time as that of acquiring the intra-oral scan data for the training purposes. The data provided by these scans are used for training the machine learning model, and more specifically, for training the machine learning model which uses the training data set. Thus, the time between the extra-oral image scan and the intra-oral scan should be as short as possible for the purpose of obtaining reliable combined extra-oral image data and intra-oral scan data.

Combining extra-oral image data with the intra-oral scan data may as previously explained, include aligning, merging or overlapping the extra-oral image data with the intra-oral scan data for the purpose of obtaining a training data set. Accordingly, the training data set may be obtained in a pre-processing step, comprising aligning the one or more periodontal structures to the intra-oral scan data by combining information from the extra-oral image data which are not part of the intra-oral scan data with intra-oral scan data as previously described. For example, the extra-oral image data may include information about teeth and bone, and the intra-oral scan data may include information about teeth and soft-tissue, and the training data set may include the information of the teeth and soft-tissue provided by the intra-oral scan data combined with bone information provided by the extra-oral image data.

In other words, combining in the context of this application further comprises the process of pre-processing the extra-oral and intra-oral data to create the training dataset from the information available in the two data types (i.e. the extra-oral image data and the intra-oral image data). Creating the training dataset comprises mapping as previously described, the extra-oral determined periodontal structures (in the form of labels or annotations) with intra-oral surface scan data to create the training data and subsequently to input the training data to the machine learning model (as previously described).

When having generated the training data set as described, the generated training data set is used to train the machine learning model to output at least the probability that a periodontal structure is identified in the input data (i.e. an intra-oral surface scan data) for one or more teeth. Thus, the input to the machine learning model is a previously unseen intra-oral scan data of a patient which is input to the trained machine learning model. The word “unseen” intra-oral scan data should be understood as data which was not used to train the machine learning model. Subsequently, the trained machine learning model is configured to process the intra-oral scan data and output a diagnostics dataset correlated with the input data. The diagnostics dataset may comprise a prediction that a periodontal structure is present on one or more of the teeth in the data set and at what location that prediction is present.

Accordingly, in view of an aspect, a computer implemented method for providing diagnostic data set of a patient based on a training data set is disclosed. The method comprising receiving intra-oral scan data of the patient, and outputting a diagnostic data set of the patient, where the diagnostic data set may be determined by processing the intra-oral scan data of the patient based on the training data set. In other words, the machine learning model may be trained on the training data set and subsequently used in a clinical practice setup, where a patient intra-oral scan not forming part of the training process is input to the previously trained machine learning model. Based on the training of the model, the machine learning model is then capable of outputting a diagnostics dataset identifying in the intra-oral scan the periodontal structures (especially bone level because of bone loss) present in the patient intra-oral scan. More specifically the diagnostic data comprises a prediction that a periodontal structure is present on one or more teeth in the intra-oral scan data and at what location that prediction is present. Accordingly, the machine learning model, when trained as described herein, eliminates the need for radiograph imaging to assess if a patient suffers from e.g. bone loss.

The term “patient” in the context of the disclosure means a person being scanned by an intra-oral scanner during a visit at a dental clinic. The intra-oral scan data provided by the scan is used as an input to the previously trained machine learning model which then processes the data based on the training data set and outputs a diagnostic data set. The diagnostic data set may include information about one or more periodontal structures, such as bone level and/or bone loss. The information may be configured to provide a diagnostical state of the one or more periodontal structures, which a dentist or a doctor may use for treatment of the patient for improving the diagnostical state of the one or more periodontal structures. The machine learning model may be represented by a deep learning model or an artificial intelligence model, such as a neural network.

Furthermore, the machine learning model includes the training data set, and the model may be configured to provide diagnostic data set of a patient by processing intra-oral scan data of the patient based on the training data set, as previously described. The machine learning model may include one or more of the following known topologies, networks or models, such as Convolutional Neural Networks (CNN), Three-circle model, persistent homology method, algebraic topology etc.

In the examples given herein, the extra-oral and intra-oral scan data is preferably image data which are input to the machine learning model. The periodontal structures determined from the extra-oral image data may be considered as labels and/or annotations as described and may be considered as annotations and/or labels aligned to the intra-oral scan data either by imaging warping techniques and/or by generating a mapping matrix comprising the information on periodontal structure and location on teeth in the data.

Accordingly, the data structures input to the machine learning model are preferably image data, which is best processed by different types of convolutional neural networks. In dependence on the task given the neural network may generally be configured to perform the task of one of image classification, object recognition and/or image segmentation.

In image classification, the training data set is configured to allow the neural network to perform a single-label classification on the input image. That is, each image in the dataset used for training has a corresponding label and/or annotation, and the model outputs a single prediction for each image it encounters. In another setup, the neural network is configured to perform a multi-label classification, where each image in the training data set has multiple labels and/or annotations allowing the neural network to output multi label predictions for each image it encounters.

In another example of object recognition, the neural network may be configured to perform an object detection task, where the neural network model is configured to detect an object and its location. With a bounding box, the target object in the image is contained within a small rectangular box accompanied by a descriptive label.

In image segmentation the neural network models use algorithms to separate objects in the image from both their backgrounds and other objects. By using labels and/or annotations to map pixels of the input image to specific features, allows the model to divide the input image into subgroups called segments. As the shape of the segments is used by the models to predict the different segments, it is important that the shapes are annotated by their shape and not only by labels.

That is, the training dataset described herein may be processed in a pre-processing step to be suitable for input to at least a convolutional neural network utilizing one of the above mentioned processing methods.

In an example using e.g. single label classification the input image to the neural network may be intra-oral surface scan together with its corresponding label as extracted from the extra-oral image data as the periodontal features present in the corresponding image. A plurality of such intra-oral scan data with its corresponding periodontal structure labels may be input to the neural network. The output of such neural network would be a single prediction of the presence of periodontal structures for each image it encounters, i.e. in each new intra-oral scan data image that the neural network encounter during a clinical visit of a patient.

In an alternative using e.g. a multi-label classification, each input image in the training data set may be an intra-oral scan data together with corresponding multiple labels of periodontal structures as extracted from the extra-oral scan data alone and/or as given by further annotations from e.g. a dentist. The multilabel configuration could also include patient specific information, such as gender, age, dental diseases etc. In this example, the neural network may output a multi label prediction for each image it encounters, that is, the output may include predictions of periodontal structures, age, gender, other diseases etc. of the input intra-oral scan images.

In the example using image segmentation, the input image may be the intra-oral scan data and corresponding labels in the form of the extra-oral image extracted periodontal features as e.g. drawn to align with the corresponding intra-oral scan data. The “drawn” periodontal features could be aligned directly with the intra-oral scan data in view of a warping process but could also be provided to the neural network as a mapping matrix. In any case, the neural network models output a segmentation map, which maps the subgroups of identified segments. The segmentation map may comprise label information, tooth information and object information, which in the example described means that the segmentation map could comprise one or more periodontal reference structures, location information and tooth information, etc.

The training data set may not only be for the machine learning model but also for an artificial learning model or a deep learning model that includes a topology for training data and providing diagnostic data.

In more detail, to generate the training data set, a pre-processing of the extra-oral image data and the intra-oral image data may be performed, as previously described. The pre-processing of the training data set may be based on receiving extra-oral image data of a plurality of candidate patients. The extra-oral image data may be collected from multiple scans of multiple candidate patients at different points in time and may be stored in an image database for further processing.

Further, in addition to receiving extra-oral image data, the method may also comprise receiving intra-oral scan data of the plurality of candidate patients, wherein the intra-oral scan data is provided by an intra-oral scanner (such as the 3 Shape A/S Trios handheld intra-oral scanner). The intra-oral scan data and the extra-oral image data is collected at substantially the same time as previously discussed to ensure that the data represent the same stage of the teeth and potential development of bone loss and/or other periodontal diseases. This ensures that the transferring of extra-oral image annotations and/or labels to intra-oral scan data represents accurately the state of the teeth in the intra-oral scan data obtained at the substantially same time.

Therefore, to create an intra-oral surface scan dataset with extra-oral periodontal structures annotated and/or labeled, the method may comprise correlating extra-oral image data with intra-oral image data of the same candidate patient taken at substantially the same point in time. In an example, each extra-oral and intra-oral dataset of a single candidate patient may be stored in the image database together with an identification tag correlating the extra-oral data, intra-oral data and the identification tag. The identification tag may be an anonymized unique tag to a specific dataset (of extra-oral and intra-oral data) of a specific candidate patient. Accordingly, a large database (comprising e.g. 500, 1000 or above samples) of extra-oral images and correlated intra-oral images of a plurality of candidate patients may be created.

Further, the extra-oral image data for use in generating the training dataset may comprise information about teeth and bone, such as information on bone level and bone loss estimates of the extra-oral image data. In accordance herewith the extra-oral image data may comprise extra-oral annotations identifying landmarks of the bone level and corresponding bone loss on the extra-oral image. Such bone landmark identification, also denoted labels herein, may be stored in the database together with the images at which they belong to. The landmark identification may be assessed on the extra-oral image for all teeth in the image and may contain values of any of the following parameters: location on a tooth, average bone loss, average bone level, buccal site, lingual site and/or, a spline representing the landmark behavior etc.

In one example, these landmarks may be annotated by e.g. human annotators presented with the extra-oral image data before creating the training dataset for the machine learning model. The annotators can mark the landmark regions in a plurality of different ways, while the processor ensures to correctly store information about location of a landmark, value, such as level or estimate of a landmark in a database together with the extra-oral image data being annotated. Accordingly, the image database also contains landmark information (also denoted information about teeth) for each of the extra-oral image data collected for the plurality of candidate patients.

In an alternative method, the landmark identification just described for the extra-oral images may be done automatically by a machine learning method trained to identify these landmarks in extra-oral image data.

To create the training dataset the extra-oral and intra-oral images stored in the database may be processed to combining extra-oral image data with the intra-oral scan data by e.g., a process of aligning, merging and/or overlapping the extra-oral image data with the intra-oral scan data for the purpose of obtaining a training data set. In more detail, each extra-oral and corresponding intra-oral image of the plurality of candidate patients in the database is input to a processor together with the extra-oral landmark identification (i.e. the information about teeth and bone) obtained from either the automatic method or the annotation method described. That is, the step of combining is performed by a processor configured to process the data from the extra-oral image and the intra-oral scan data to ensure that the extra-oral generated annotations are merged (such as transferred) to the intra-oral image data.

In one example, the method comprises to read into the processor the extra-oral image data and corresponding landmark information, and to read into the processor the intra-oral image data. Subsequently transform the landmark information data from the extra-oral image data into a data representation (e.g. a matrix representation) comprising e.g. tooth no. information, identification of a periodontal structure landmark in the form of yes or no, identification of site (i.e. buccal or lingual site) of the tooth at which the periodontal structure is identified and/or at which one or more of one out of three sites on each of the buccal and lingual site that the periodontal structure landmark is present. Further information such as age, gender etc. of the patient may also be represented.

The processor is subsequently configured to transfer the data representation to the intra-oral scan data to generate the training data set for use in training the machine learning model. In this way a training dataset combining the extra-oral image data with the intra-oral scan data for each of the plurality of candidate patients for aligning the one or more periodontal structures to the intra-oral scan data is generated. The training data set may subsequently be used as an input to a machine learning model for training the machine learning model. The machine learning is configured to learn from the training data set when a given structure in an input data set (such as an intra-oral scan data obtained of a patient in a clinical session) contains a periodontal structure of interest. Accordingly, the machine learning model may be configured to e.g., assign a probability value (e.g. a numerical value ranging from 0 to 1, where a larger value is associated with greater confidence) that a periodontal feature exists to each pixel in a given input data in the form of an intra-oral scan data not forming part of the training dataset. The machine learning model may be configured, as previously described, to perform single or multilabel classification, image segmentation and/or object recognition.

The intra-oral scan data can then be post-processed into various forms to be displayed in e.g. a graphical user interface. Accordingly, the machine learning model outputs a diagnostic data set comprising a set of probability scores, an object recognition map and/or a segmentation map for presence of detected periodontal structures and/or a map encoding the location of any detected periodontal structure in the intra-oral scan data. By using back-propagation to the output from the machine learning model it is possible to visualize the output from the machine learning model on the previous unseen image input. Thus, it is possible to visualize the input image of e.g. an upper or lower jaw and fuse onto the image by e.g. image warping techniques the output from the neural network to efficiently illustrate to a user the findings of periodontal structures at the specific input image.

Accordingly, by using the trained neural network (as described herein) on new image data it is possible to assess without the use of radiation if the patient suffers from periodontal disease. In dependence of the neural network used and the input data that the neural network has been trained on, the assessment of periodontal disease may include identification of the location on a tooth where a bone loss is present, the amount of bone level for a specific tooth, the age of the person, the gender of the person, potential other disease related to specific teeth of the input image data as acquired from an intra-oral scanner.

As already mentioned, the methods described herein may also use more specific patient information as input to the neural network. This potentially improves the reliability of the method, as the method may further comprise receiving candidate patient information of the plurality of candidate patients and generating the training data set by combining the received patient information for each of the plurality of candidate patients with the combined extra-oral image data and the intra-oral scan data for each of the plurality of candidate patients. The candidate patient information may be age, gender, health condition, diseases, ethnicity etc. By applying the candidate patient information into the method may provide the possibility in grouping the training data set into one or more of the candidate patient information. The training of the training data set may be done in parallel for each of the group of training data set. Each group of the training data set may be labelled by one of the gender information. This improves the accuracy and the reliability of the method and the machine learning model.

The method may include segmenting the extra-oral image data of the plurality of candidate patients into teeth and bone based on an extra-oral segmentation algorithm and segmenting the intra-oral scan data of the plurality of candidate patients into teeth and soft tissue based on an intra-oral segmentation algorithm. Segmentation of data is known and would not be described further in this disclosure. However, examples are given as follows. The extra-oral segmentation algorithm may be configured to perform segmentation of data provided by an extra-oral image device, and the intra-oral segmentation algorithm may be configured to perform segmentation of data provided by an intra-oral scanner. The segmentation process may comprise to segment the extra-oral image and/or intra-oral image data into a number of segments representing object types for example by applying pixel based segmentation technique, block based segmentation technique, or any other conventionally known segmentation technique, selecting a segment from the number of segments that is associated with a plurality of distinct features; compute an aggregation of the plurality of distinct features to generate a single feature describing the object type, and recognizing a type of the object based upon the aggregation by comparing the aggregation to features corresponding with known object types from the object-type specific trained data set. In this way teeth and bone in view of the extra-oral image data and gingiva and teeth in view of the intra-oral image data may be separated from each other.

In another example, the segmentation of the extra-oral image data and the intra-oral scan data may be provided by a segmentation algorithm configured for both types of scanning, i.e. intra-oral scanning and extra-oral imaging. The segmentation provides the ability to distinguish between teeth and bone in the extra-oral image data and to distinguish between teeth and soft tissue in the intra-oral scan data. This improves the reliability of the training data set and the accuracy of the diagnostic data. Without the segmentation, the model may combine and align the extra-oral image data and the intra-oral scan data based on random data used as reference points or surfaces.

Accordingly, the pre-processing step may also comprise the described step of segmentation of the extra-oral image data into teeth and bone. This may allow the creation of a 2D surface representation of the extra-oral image data, where the bone level and corresponding bone loss may be assessed. Accordingly, the extra-oral image data to be used in the training data set may be raw extra-oral image data and/or a segmented extra-oral image data. In both cases, the bone level information for candidate patients may be assessed by a dental practitioner in a manual annotation process and/or identified e.g. using a trained neural network configured to identity bone level in extra-oral image data.

The combining of the extra-oral image data and the intra-oral scan data may in one example use the segmented teeth of the extra-oral image data and the segmented teeth of the intra-oral scan data as reference surfaces. The combining of the extra-oral image data and the intra-oral scan data may be based on an alignment of the intra-oral scan data with the extra-oral image data using the segmented teeth of the extra-oral image data and of the intra-oral scan data as reference surfaces. Both the extra-oral image device and the intra-oral scanner provide reliable scan data of teeth and using the segmented teeth as reference surfaces will result in an even more reliable training data set and an even more accurate diagnostic data set. The extra-oral image device is not able to provide reliable soft tissue data and the intra-oral scanner is not able to provide reliable bone data, and if using either the segmented soft tissue or the segmented bone as the reference surfaces will result in less reliable training data and less accurate diagnostic data in relation to the previous example. In other words, the extra-oral image data is suitable for obtaining periodontal structure information, such as information about bone, such as bone level and estimated bone loss, whereas the intra-oral scan data is suitable for obtaining soft tissue diagnostics, such as recession of the gum, redness of the gum etc. Using both types of data in pre-processing of the data to generate the training data to be used in the machine learning model ensures that periodontal structures which may normally not be identified in an intra-oral scan data can be identified in such data.

Further, if in addition, using the information of the soft tissue from the intra-oral scan data, an estimate of the development of more periodontal diseases can be assessed. That is, the intra-oral scan data may provide information on e.g. gingival recession and gingivitis, which is at least one clinical precursor for development for example bone loss. From a clinical aspect, inflammation, such as gingivitis and correlated gingival recession are usually the major cause of teeth bone loss. Thus, inflammation and changes occurring in the soft tissue which may be identified in an intra-oral scan may be correlated with bone loss as identified from the extra-oral image data. Accordingly, a relationship between soft tissue findings (i.e. in intra-oral scan data) and hard tissue findings (i.e. in extra-oral scan data) may be defined to train the neural network to be able to estimate e.g. average values of bone loss pr. tooth from the extra-oral image data and correlated average values of e.g. gingival recession and/or gingivitis from the intra-oral scan data. In this way, a mathematical relationship correlating the finding of bone loss with gingival recession or gingivitis may be created. Such mathematical relationship may be input as a parameter in the form of e.g. a label to the machine learning model in the training process, creating a machine learning model capable of outputting an average estimate of bone loss and associated gingival recession and/or gingivitis in the intra-oral scan data.

Based on the segmented teeth data which is provided by the segmentation of the intra-oral scan data and/or the extra-oral image data a cementoenamel junction may be determined for each segmented tooth. The cementoenamel junction (CEJ) represents the anatomic limit between a crown and a root surface of a tooth and is defined as the area of union of the cementum and enamel at a cervical region of a tooth. A bone edge may be determined based on the segmented bone which is provided by the segmentation of the extra-oral image data. The determining of the one or more periodontal structures includes a bone level which is determined for each of the segmented teeth in the extra-oral image data by a distance between the bone edge determined by the segmented bone and the cementoenamel junction, and where the cementoenamel junction is determined by the segmented teeth. In other words, the computer implemented method may comprise determining of the one or more periodontal structures includes determining a bone level for each of the teeth in the extra-oral image data, wherein the bone level is determined by a distance between an identified bone reference point and an identified cementoenamel junction of the extra-oral image data, and wherein the determined bone level for the extra-oral image data is merged with the intra-oral scan data. A cementoenamel machine learning algorithm may be configured to determine the cementoenamel junction based on the extra-oral image data. The combining of the extra-oral image data with the intra-oral scan data may include an alignment of the determined distance for the extra-oral image data with the intra-oral scan data. Accordingly, the segmentation of extra-oral image data may assist in e.g. performing labeling of the data from an automated process and or manually as done by a dental practitioner. The identification of the CEJ may be input as a label to the machine learning model to ensure that the machine learning model is capable of predicting the position of the CEJ in a new unseen intra-oral scan data. Accordingly, the CEJ is considered to form part of the periodontal structures previously described. Also, the mentioned bone edge may be considered as a label and/or annotation identified in the extra-oral image data as a periodontal structure to be input the to machine learning model.

The determining of the one or more periodontal structures for each of the plurality of candidate patients may include determining a first relation between the segmented teeth and bone of the extra-oral image data using the one or more reference sites per identified tooth; and combining the extra-oral image data with the intra-oral scan data by aligning the first relation with the intra-oral scan data. The first relation may comprise a distance between a tooth reference point arranged on a tooth of the extra-oral image data (potentially a segmented teeth) and a bone reference point arranged on a bone which is closest to the tooth reference point or at least in vicinity to the tooth reference point. This first relation may be considered as a label of periodontal structures which are input to the machine learning model in the training phase.

The determining of the one or more periodontal structures for each of the plurality of candidate patients includes identifying in the extra-oral image data a plurality of teeth; and determining one or more reference sites per identified tooth of the extra-oral image data; and determining the one or more periodontal structures at each of the one or more reference sites. In one embodiment the determination of the periodontal structures may be found on a dataset comprising a segmentation of the extra-oral image data into segmented teeth of the extra-oral image data.

The one or more reference sites may include a tooth reference point, a bone reference point, and/or a soft tissue point as previously described. Accordingly, in one embodiment the method comprises determining for each of the reference site a tooth reference point and a bone reference point using the extra-oral image data. All of these mentioned reference sites may be considered as a label and/or annotation suitable for inputting to the machine learning model as described herein. A reference site may indicate where the one or more periodontal structures have been determined, i.e. a location of a tooth where a periodontal structure is found. For improving the accuracy of determining one or more periodontal structures around and/or at a tooth at least three reference sites may be used, and more ideally, around 6 reference sites. The reference sites may be arranged around at least a tooth of the segmented teeth. In more detail, at least 3 sites on the buccal side of a tooth and at least 3 sites on the lingual side of a tooth is preferred. This ensures that a potential condition, such as bone loss can be identified on all sides of a tooth to evaluate the level of the condition development across the entire tooth. Accordingly, when pre-processing the data for training of the machine learning model, an annotation and/or label on the extra-oral image data may be given for all 6 sites of the tooth so as to form labels to be input to the machine learning model in the training phase. These labels may be correlated with intra-oral scan data as previously described, e.g. in a matrix transformation.

It should be noted that in the context of the application, a tooth reference point may be the placement of the CEJ as identified from extra-oral image data and the bone reference point may be e.g. a bone edge as identified from the extra-oral image data, each of the tooth reference points and/or bone reference points being considered as labels used as input to the machine learning model.

For improving the accuracy and the reliability of the method and the machine learning model the training data set may be customized for a patient. The method may further comprise receiving intra-oral scan data of the patient at a first time and within a time-period and customizing the training data set to the patient by including the intra-oral scan data of the patient. In other words, the customization may be configured as an update of the already once trained machine learning model to include information gathered from a patient scan at a clinical visit. In this way, the clinical data, that a dentist identifies in the intra-oral scan of the patient may be directly transferred to the machine learning model to update the training data with the new information. In this way the machine learning model is constantly updated with new information and will learn further clinical aspects of the dental conditions automatically. The customization described throughout may generally be considered as an automatic update of the previously trained neural network with additional information as acquired during e.g. further scanning of patients and or if acquiring new extra-oral image data. The customization may comprise utilizing un-supervised learning, where the neural networks used are configured to automatically update the network without having the knowledge about labels and annotations.

For improving even more, the accuracy and the reliability of the method and the machine learning model, the training data set may be customized for the patient by receiving intra-oral scan data of the same patient at a second time and within the time-period. The improvement will continue as long as the method and the machine learning model receives more intra-oral scan data of the same patient within the time-period.

For improving even more, the accuracy and the reliability of the method and the machine learning model, the training data set may be customized for the patient by receiving multiple of intra-oral scan data of the same patient, where the intra-oral scans for generating the plurality of intra-scan data are performed over a period. The multiple intra-oral scan data received over time provides reliable detection of bone loss over time, as it is possible to detect bone level differences between two or more scans.

The machine learning model may be configured to generate a first training data set configured for single intra-oral scan for each of the plurality of candidate patients and a second training data set configured for multiple scans for each of the plurality of candidate patients. For example, if multiple intra-oral scans are being performed on the same patient, the machine learning model is then configured to use the second training data set for providing diagnostic data set.

The method may further comprise receiving intra-oral scan data of a patient at a first time and within a time-period, receiving intra-oral scan data of the patient at a second time and within the time-period, aligning the intra-oral scan data received at the first time and at the second time, and customizing the training data set to the patient based on a difference in the alignment of the intra-oral scan data received at the first time and at the second time. The multiple intra-oral scan data received over time (e.g. at the first time and the second time) provides reliable detection of bone loss over time, as it is possible to detect bone level differences between two or more scans. At least it is considered to be possible to detect if bone loss is present in this way.

For improving even more, the accuracy and the reliability of the method and the machine learning model it will be beneficial to distinguish between scan data that the method should use for training the training data set. For example, the scan data may be of a low quality which will lead to an erroneous training of the training data set, and thereby, result in less accurate and reliable method. Therefore, it will be an advantage if the method further comprises determining whether to customize the training data to the patient based on one or more criteria, such as quality of the intra-oral scan data of the patient, and level of mismatch between intra-oral scan data of the patient and the training data set.

In one embodiment, the machine learning model which includes the training data may be configured to provide a diagnostic data set of the patient by processing intra-oral scan data of the patient based on the training data set. The method may further comprise receiving patient information of the patient, and the diagnostic data set may be determined by processing the intra-oral scan data and the patient information of the patient based on the training data set which also includes patient information of the plurality of candidate patients. Including the patient information will inevitably result in more accurate diagnostic data set. The diagnostic data set may include one or more periodontal structures determined by processing the intra-oral scan data of the patient based on the training data set.

For improving the accuracy of the diagnostic data set the method may further comprise determining the periodontal structures by receiving the intra-oral scan data of the patient at a first time and within a time-period, segmenting the intra-oral scan data received at the first time into teeth and soft tissue based on an intra-oral segmentation algorithm, aligning the segmented teeth of the intra-oral scan data received at the first time with segmented teeth of the training data set, retrieving from the training data set at least the segmented bone corresponding to the segmented teeth of the training data set, combining the segmented bone with the segmented teeth of the intra-oral scan data received at the first time, and determining the one or more periodontal structures based on retrieved segmented bone and the segmented teeth of the intra-oral scan data of the patient.

For improving even more the accuracy of the diagnostic data the method may further comprise receiving intra-oral scan data of the patient at a second time and within the time-period, segmenting the intra-oral scan data received at the second time into teeth and soft tissue based on the intra-oral segmentation algorithm, aligning the segmented teeth of the intra-oral scan data received at the second time with segmented teeth of the training data set, determining a difference in an alignment of the segmented teeth and soft tissue of the intra-oral scan data received at the first time and at the second time, retrieving from the training data at least the segmented bone corresponding to the determined difference in the alignment, combining the segmented bone with the segmented teeth of the intra-oral scan data received at the second time and determining the one or more periodontal structures based on the retrieved segmented bone and the segmented teeth of the intra-oral scan data of the patient. By receiving multiple intra-oral scan data at different times and within the time-period will result in a more customized training data set which at the end will result in a more accurate diagnostic data set. The time-period should be more than days, weeks or years. The time between the first and the second time should be more than days, weeks or years.

The determining of the one or more periodontal structures of the patient may include determining one or more reference sites per identified tooth of the segmented teeth of the intra-oral scan data received at the first time or the second time and determining the one or more periodontal structures of the patient by correlating the intra-oral scan data received at the first time or the second time with the training data set at each of the one or more reference sites.

The determining of the one or more periodontal structures at each of the one or more reference sites may comprise identifying a cementoenamel junction for each of the segmented teeth of the intra-oral scan data of the patient, determining a distance between the cementoenamel junction and the segmented tissue around each of the segmented teeth, and correlating the distance with the training data set for determining the one or more periodontal structures.

The determining of the one or more periodontal structures at each of the one or more reference sites may include: determining a relation between the segmented teeth and soft-tissue of the patient, correlating the relation with the training data which includes a correlation of a relation between the segmented teeth and the segmented bone provided by the extra-oral image data of the plurality of candidate patients and a relation between the segmented teeth and the segmented soft tissue provided by the intra-oral scan data of the plurality of candidate patients.

The one or more periodontal structures as identified using the trained machine learning model may be presented on a graphical user interface which may include presenting a 2D model or a 3D model of a mouth anatomy together with the identified periodontal structures as determined by the diagnostic data. Accordingly, the analyzed data of the intra-oral scan input to the machine learning model can be used in many ways: First, one or more presentation techniques may be employed by the computer system to present the analyzed data; for example various types of user interfaces, graphical representations, etc., may be used to efficiently present the data and quickly alert the professional to potential areas of interest signaling on the monitor potential detected features which need immediate attention by the user. In another example, the graphical user interface may include a dental chart on which the one or more periodontal structures identified from the machine learning model are visualized.

The one or more periodontal structures may be visualized at the one or more reference sites. The one or more periodontal structures may be visualized with color or text. The one or more periodontal structures may be visualized if fulfilling a visualization criterium. The visualization criterium may define that the bone level is above a threshold level, and the threshold level may be between 2 mm and 3 mm, around 3 mm, or above 3 mm. The bone level may be determined between CEJ and the bone, e.g. a bone edge identified via the segmented bone.

The machine learning model described herein may be used to provide the diagnostic dataset, when having been trained on the training data as described herein. The trained machine learning model can be used in different clinical flows, as previously touched upon. Firstly, the machine learning model can be used in a cross-sectional approach, where a patient is being scanned with an intra-oral scanner at a first clinical visit. In this case, the computer-implemented method described herein provides a diagnostic data set of the patient by receiving the intra-oral scan data of the patient; inputting the intra-oral scan data to the trained machine learning model as described herein; and receiving from the trained machine learning model a diagnostic data set of the patient. The diagnostic dataset as output from the machine learning model may comprise one of the previously mentioned probability maps, segmentation maps etc. describing a prediction that the input intra-oral scan data comprises one or more periodontal structures. This diagnostic dataset may be visualized as previously described.

In a second use of the machine learning model, the patient is being scanned at a first visit as previously described and again at a second clinal visit, wherein the first visit is at a first time being different from the second visit at a second time. At both visits the obtained intra-oral scans are input to the machine learning model to create a diagnostic dataset reflecting the periodontal situation at the respective visits. Accordingly, at the second visit, two diagnostics dataset obtained at two different times is present in a data memory. To assess if a change in the periodontal structures has occurred from the first visit to the second visit at least the two diagnostic data set are compared to each other. This may provide a comparison diagnostic data set which describes the changes and potential progression in any of the identifies periodontal structures of the patients second scan at the second time.

In an example, the second scan data obtained at the second time may be used in the previously described customization of the machine learning model.

In a further example, the machine learning model may also be trained on the basis of the comparison between the first and second visit scans and diagnostic dataset outputs to be able to output directly via the diagnostic dataset a periodontal change.

The method and the machine learning model may be configured to be implemented into a computable device, such as laptop, PC, tablet, smartphone etc.

A computer program may comprise instructions which, when the program is executed by a computer, cause the computer to carry out the method.

A dental scanning system may comprise means for carrying out the method. The extra-oral image data of the plurality of candidate patients and/or the intra-oral scan data of the plurality of candidate patients may be stored on a server, a memory of the computer, or a cloud server part of the dental scanning system. The dental scanning system may include an extra-oral image device interface configured for receiving the extra-oral image data, and the system may further include an intra-oral scanner interface configured for receiving the intra-oral scan data. Both interfaces may share one interface. The interface may be wired and/or wireless. The training data set may be stored on the server, the memory of the computer, or the cloud server. The computer may be configured to execute operations on one or more artificial techniques, including one or more machine learning models, artificial networks, can be used to analyze the data of the combined extra-oral and intra-oral data and present resulting output.

A data processing device may comprise means for carrying out the method.

A computer-readable storage medium may comprise instructions which, when executed by a computer, cause the computer to carry out the method.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with a reference to the illustrations described hereinafter in which:

FIGS. 1A and 1B illustrate a computer implemented method;

FIG. 2 illustrates a method including a machine learning model;

FIGS. 3A-3D illustrate different examples on how the method generates training data set or diagnostic data;

FIG. 4 illustrates an example of the method where the training data set is being customized;

FIG. 5 illustrates an example of the method where the training data set is being customized;

FIG. 6 illustrates another example of the method;

FIGS. 7A-7C illustrate another example of the method;

FIGS. 8A-8C illustrate a graphical visualization of periodontal structures;

FIG. 9 illustrates a dental scanning system;

FIG. 10 illustrates the combining of the extra-oral and intra-oral data to create the training data set;

FIG. 11 illustrates the machine learning model used for the training phase, and

FIG. 12 illustrated use if the trained machine learning model at two different clinical visits to determine a change in periodontal structures over time.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing extra-oral image data may comprise a scanner which is based on an ionizing radiation technology, such as an X-ray or CBCT scanner. The intra-oral scan data may be obtained by a dental scanning system that may include an intraoral scanning device such as the TRIOS series scanners from 3Shape A/S (i.e. a handheld intra-oral scanner) or a laboratory-based scanner such as the E-series scanners from 3Shape A/S. The scanning devices described herein may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching in the post-processing. The result of stitching may be the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

The scanning device, especially the intra-oral scanning device may comprise one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety. Accordingly, the intra-oral scanning device described herein is preferably a handheld intra-oral scanner which may be operated by a dentist to scan a patients oral cavity including teeth, during a clinical visit.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The dental scanning system preferably further comprises a processor configured to generate scan data (such as extra-oral image data and/or intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

As is apparent, the scanning devices described herein, such as the extra-oral scanning device and the intra-oral scanning device may be used to generate extra-oral image data and intra-oral scan data, respectively. As will be understood, the extra-oral image data may be used for generating a training data set together with intra-oral scan data obtained substantially at the same time as the extra-oral image data. The training data set may be generated in a training phase and may include one or more preprocessing steps configured to adjust the data to a data format suitable for inputting to a machine learning model as will be described in the following.

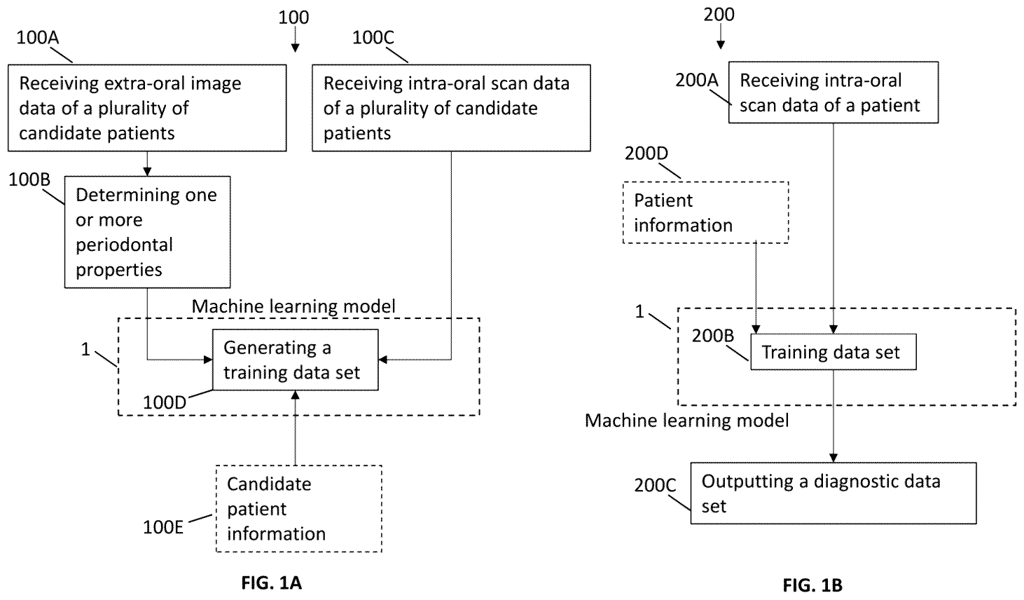

FIGS. 1A and 1B illustrate a computer implemented method (100,200) for generating a training data set for a machine learning model and for providing a diagnostic data set of a patient based on the training data set, respectively. FIG. 1A illustrates a computer-implemented method 100 of generating a training data set for a machine learning model 1, wherein the method comprising; receiving 100A extra-oral image data of a plurality of candidate patients provided by an extra-oral image device; determining 100B one or more periodontal structures (denoted periodontal properties in FIG. 1A) for each of the plurality of candidate patients based on the received extra-oral image data; 100C receiving intra-oral scan data of the plurality of candidate patients provided by an intra-oral scanner, and generating 100D a training data set by combining the extra-oral image data with the intra-oral scan data for each of the plurality of candidate patients for aligning the one or more periodontal structures to the intra-oral scan data.

As previously mentioned, combining is considered to comprise the process of image alignment, where elements in one image is mapped into meaningful correspondence with elements in a second image. In other words, the combining of the extra-oral image data with the intra-oral scan data comprises merging the data structures together to transfer information gathered from the extra-oral dataset to the intra-oral dataset. In the context of machine learning, it should be understood that the extra-oral data information may be used as annotating or/and labeling the intra-oral dataset with the determined or more periodontal structure information. That is, as the intra-oral scan data does not provide information about periodontal structure, it would not be possible to use the intra-oral scan data only for the training of the dataset. Instead, the extra-oral scan data is collected to gather information about periodontal structures to be able to annotate and/or label the intra-oral scan data with that information.

Alternatively, the method 100 further comprises receiving 100E candidate patient information of the plurality of candidate patients and generating the training data set by combining the received patient information for each of the plurality of candidate patients with the combined extra-oral image data and the intra-oral scan data for each of the plurality of candidate patients.

FIG. 1B illustrates a computer implemented method 200 that comprises receiving 200A intra-oral scan data of the patient and processing 200B the intra-oral scan data of the patient based on the training data set. The method 200 further comprises outputting 200C the diagnostic data. Accordingly, FIG. 1B illustrates the situation, where the machine learning model after having been trained according to FIG. 1A is used in a clinical practice setup. Here the machine learning model is utilized by a computer to provide a diagnostic dataset from a unseen intra-oral scan data obtained by scanning a patient during a clinical visit. In this FIG. 1A it is illustrated how the diagnostic dataset is obtained by the method by receiving the intra-oral scan data of the patient; inputting to the trained machine learning model the intra-oral scan data; and receiving (in the form of an output) from the trained machine learning model a diagnostic dataset of the patient.

The diagnostic data may be visualized on a graphical user interface or further analyzed by another processing unit.

In a further embodiment, illustrated in FIG. 1B, the method further comprises receiving 200D patient information, and the diagnostic data set is determined by processing 200B the intra-oral scan data and patient information of the patient based on the training data set.

As described herein, the outputting of the diagnostic data set of the patient includes one or more periodontal structures determined by the processing of the intra-oral scan data of the patient based on the training data set forming the machine learning model. Accordingly, the diagnostic dataset comprise a prediction that a periodontal structure is present on one or more teeth in the intra-oral scan data and at what location that prediction is present.

The periodontal structures may include a determination of bone level or bone loss of the plurality of candidate patients.

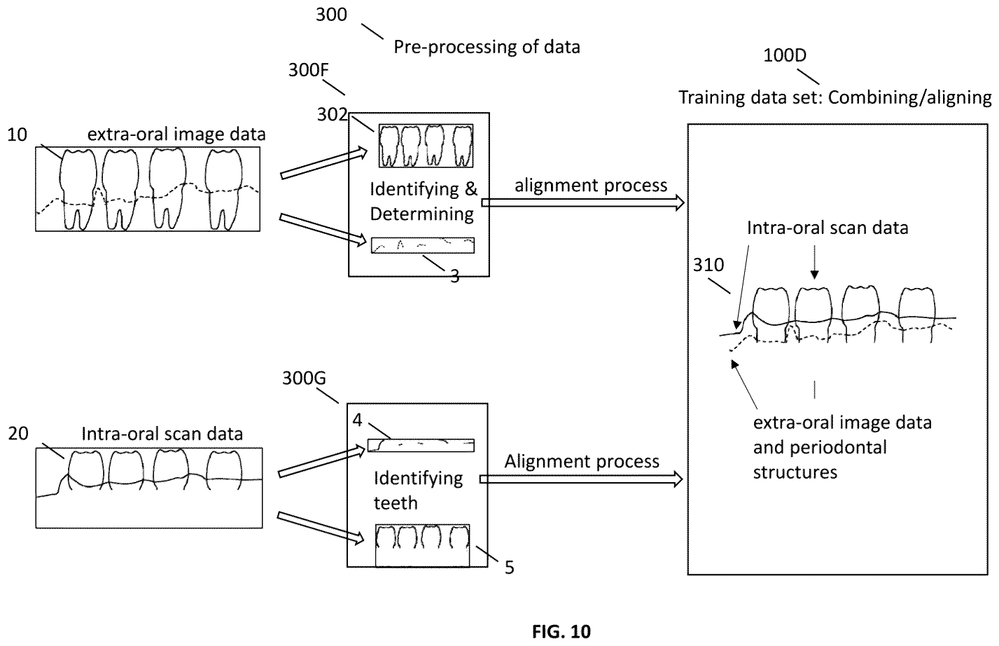

Turning now to FIG. 10, the pre-processing 300 of the extra-oral image data and the intra-oral image data to be used for training the machine learning model is illustrated in more detail. Here it is illustrated that the extra-oral image data 10 comprises bone, root and teeth information, whereas the intra-oral image scan data 20 comprises only surface information, in the form of surface of teeth and gingiva. According to the pre-processing as described herein, the two data composition (i.e. information gathered from the two different datasets) may be combined to form the training data set 100D of the machine learning model. That is, the pre-processing step comprises to combine the two data types by: identifying 300F, 300G corresponding teeth in the extra-oral image data and the intra-oral scan data; and determining in the extra-oral image data 302 one or more periodontal structures 3; and using an image alignment process to map or correlate the determined periodontal structures with corresponding teeth in the intra-oral scan data. The alignment process is illustrated in FIG. 10 as an image warping process for the purpose of illustration only. In the image warping the two datasets are merged together as seen in 310 in FIG. 10. However, as previously described this warping may not necessarily be needed as the information may be contained in a mapping matrix which is not necessarily illustrated.

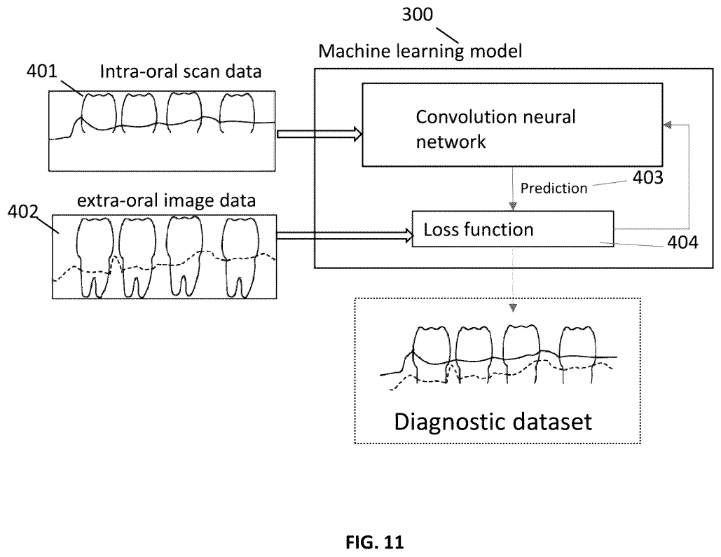

In any case, what should be understood is that the periodontal structures (given as an example as the dotted line in FIG. 10, may be used as an annotation and/or label as input to the machine learning model. Accordingly, as illustrated in FIG. 11, the machine learning model 300 during the training phase is configured to take an intra-oral scan data 401 as input to e.g. a convolutional neural network. The convolutional neural network then generates a prediction 403, which is input to a loss function 404. The loss function at the same time receives the extra-oral scan data 402 as annotation and/or labels which extra-oral scan data 402 provides to the loss function, the ground truth that the neural network should be able to estimate. In dependence on how the machine learning model managed to predict the ground truth, the machine learning model is updated until a satisfying prediction is achieved. This process in the training phase is run on all the candidate patient data forming part of the training data set. When a satisfactory prediction is obtained by the machine learning model, the model is able to predict a diagnostic dataset on a new unseen intra-oral scan data as seen in the dotted box in FIG. 11.

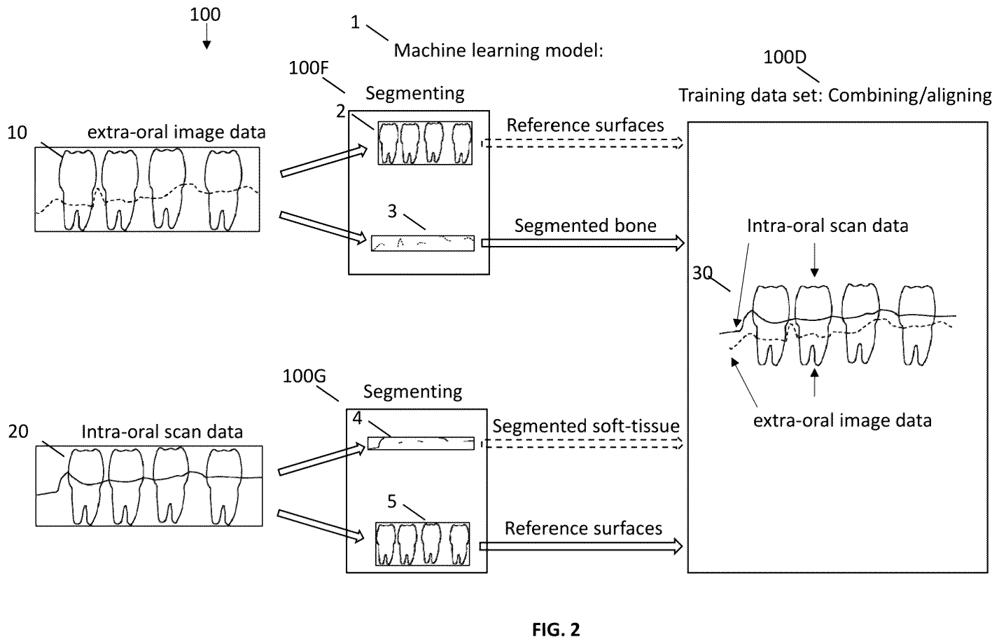

FIG. 2 illustrates an example of the method 100 including the machine learning model 1. In this example, the method comprises segmenting 100F the extra-oral image data 10 of the plurality of candidate patients into teeth 2 and bone 3 based on an extra-oral segmentation algorithm and segmenting 100G the intra-oral scan data 20 of the plurality of candidate patients into teeth 5 and soft tissue 4 based on an intra-oral segmentation algorithm. In this example, the segmented teeth (2,5) are used as reference surfaces for the combining or alignment 100D of the extra-oral image data 10 and the intra-oral scan data 20. Furthermore, in this example, the extra-oral image data 10 includes segmented bone information 3 which is not part of the intra-oral scan data 20, so the training data set 30 includes data that relates to the segmented bone 3 aligned with the segmented soft tissue 4 and the segmented teeth 5 of the intra-oral scan data 20 of the plurality of candidate patients.

In the examples given, it is possible to create a training data set comprising extra-oral image data, i.e. the periodontal structures identified in the extra-oral image data, as annotations and/or labels to the intra-oral scan data. This ensure that the machine learning model can be trained on intra-oral scan data while being able to detect periodontal structures. This would not have been possible from intra-oral scan data alone, but with the combining of extra-oral information to the intra-oral scan data this is made possible.

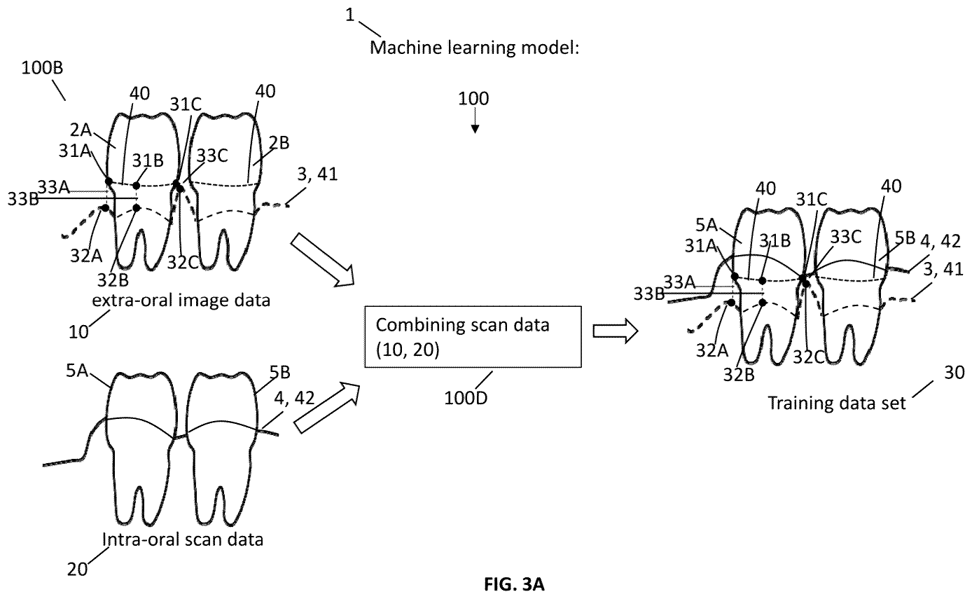

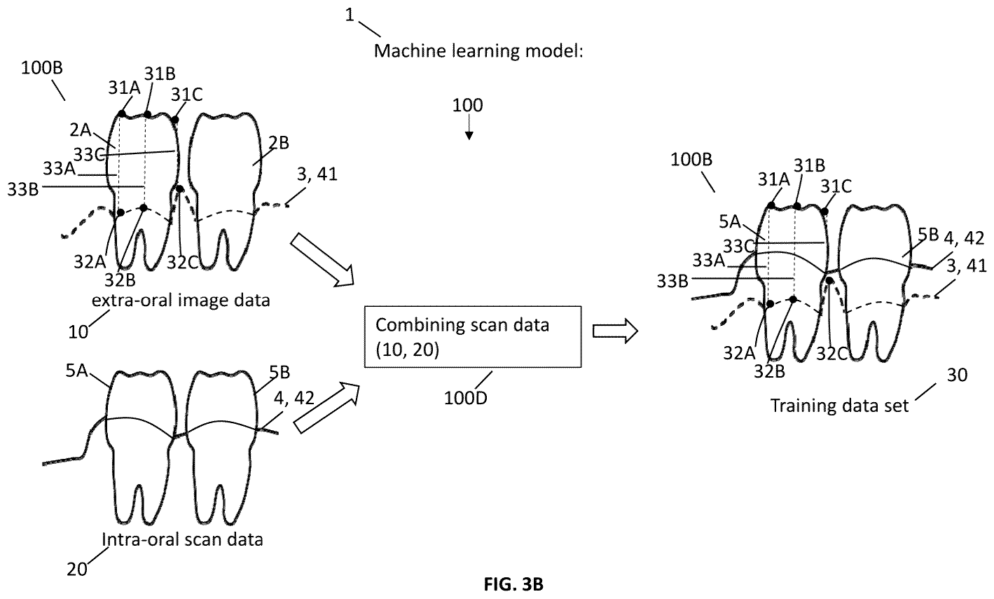

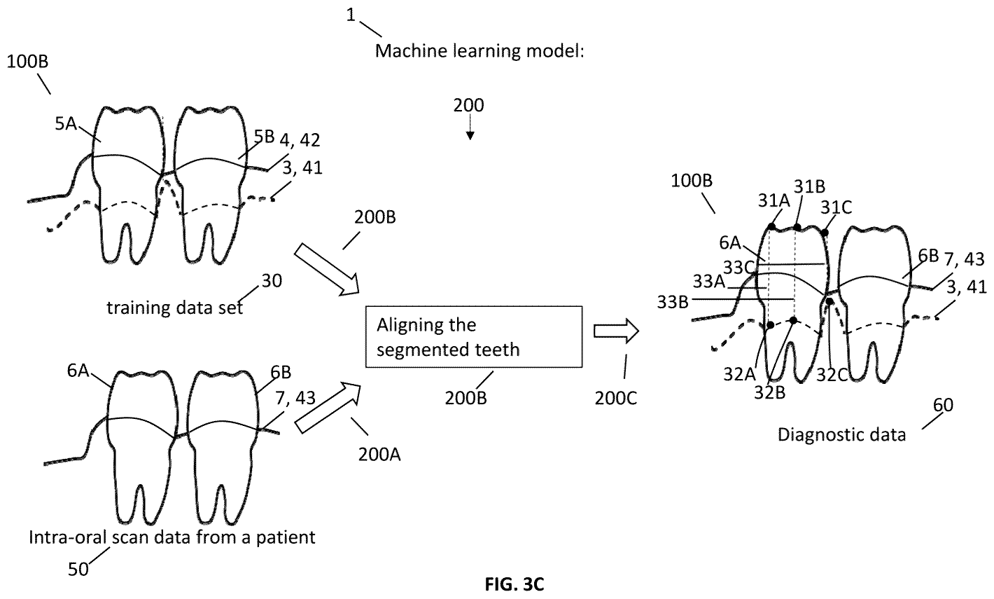

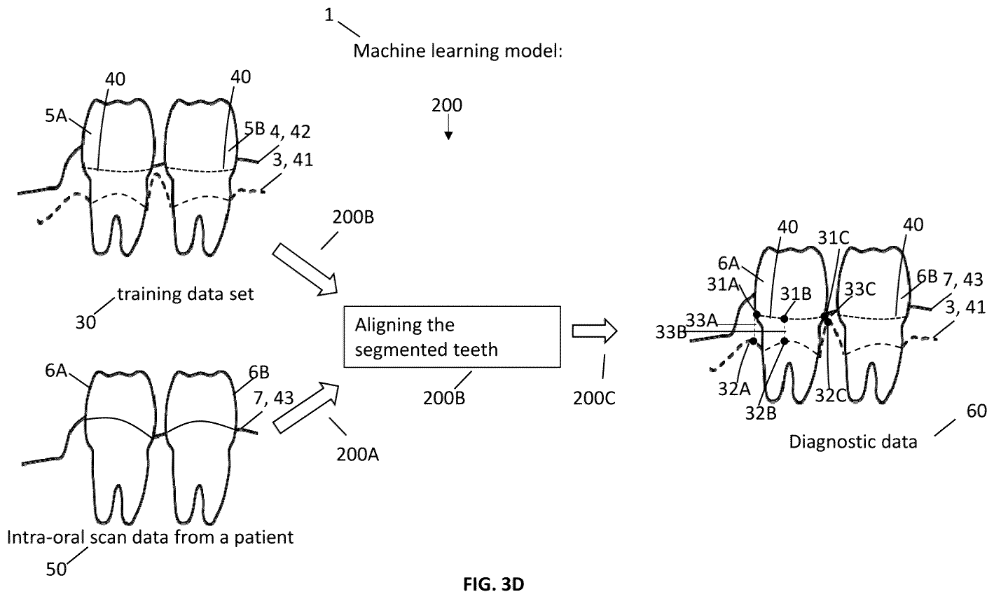

FIGS. 3A and 3B illustrate different examples on how the method 100 generates the training data set by aligning the one or more periodontal structures to the intra-oral scan data of the plurality of candidate patients. As seen from FIGS. 3A and 3B the determining of the one or more periodontal structures for each of the plurality of candidate patients comprises identifying in the extra-oral image data a plurality of teeth; determining one or more reference sites per identified tooth of the extra-oral image data; and determining the one or more periodontal structures at each of the one or more reference sites, all of which will be described in more detail in the following. FIGS. 3C and 3D illustrate different examples on how the method 200 may generate the diagnostic data.

In FIG. 3A, an example is illustrated where the extra-oral image data 10 is segmented into segmented teeth (2A, 2B) and segmented bone 3 which at least includes a bone edge 41. In this specific example, each of the segmented teeth (2A, 2B) has a cementoenamel junction 40 identified by performing a surface analysis of the tooth (2A, 2B). At least three reference sites (31A, 31B, 31C) have been determined and arranged on the cementoenamel junction 40, and further three reference sites (32A,32B, 32C) have been arranged on the bone edge 41, i.e. on the segmented bone 3. In this example, the one or more periodontal structures includes a bone level which is determined for each of the segmented teeth of the extra-oral image data 10 by determining a distance (33A, 33B, 33C) between the bone edge 41 and the cementoenamel junction 40. In this example, three distances (33A, 33B, 33C) are determined between the three references sites (31A, 31B, 31C) and the other three reference sites (32A, 32B, 32C). The combining 100D of the extra-oral image data 10 with the intra-oral scan data 20 includes combining the segmented bone 3 with the segmented teeth (5A, 5B) and the segmented soft tissue 4 which then results in an alignment of the bone level to the segmented teeth (5A, 5B) and the segmented soft tissue 4. If one or more of the distances (33A, 33B, 33C) are above 1 mm, above 2 mm, above 3 mm, above 4 mm or above 5 mm a bone loss is diagnosed. In this example, the one or more periodontal structures include both determined bone level and bone loss. Accordingly, as seen in FIG. 3A, the method comprises determining for each reference site (31A, 31B, 31C) a tooth reference point (40) and a bone reference point (41). In this example, for each of the reference sites (31A, 31B, 31C) a first relation between teeth and bone in the extra-oral image data is determined, wherein the first relation comprises a distance (33A, 33B, 33C) between the tooth reference point (i.e. CEJ) (40) and bone reference point (i.e. bone edge) (41). The determined distance, in this example, three distances (33A, 33B, 33C) constitutes the bone level for each of the reference sites.

In another example, in FIG. 3B, the determining of the one or more periodontal structures for each of the plurality of candidate patients includes determining a first relation between the segmented teeth (2A, 2B) and the segmented bone 3 of the extra-oral image data 10. In this example, the extra-oral image data has been segmented into segmented bone 3 and segmented teeth (2A, 2B), and the segmented bone 3 includes at least a bone edge 41. At least three reference sites (31A, 31B, 31C) have been determined and arranged on a tooth 2A of the segmented teeth (2A, 2B), and further three reference sites (32A, 32B, 32C) have been arranged on the bone edge 41, i.e. on the segmented bone 3. In this example, the one or more periodontal structures includes a bone level which is determined for each of the segmented teeth of the extra-oral image data 10 by determining a distance (33A, 33B, 33C) between the bone edge 41 and the tooth 2A. In this example, three distances (33A, 33B, 33C) are determined between the three references sites (31A, 31B, 31C) and the other three reference sites (32A, 32B, 32C). The combining 100D of the extra-oral image data 10 with the intra-oral scan data 20 includes combining the segmented bone 3 with the segmented teeth (5A, 5B) and the segmented soft tissue 4 which then results in an alignment of the bone level to the segmented teeth (5A, 5B) and the segmented soft tissue 4.

For both examples, if one or more of the distances (33A, 33B, 33C) are above a threshold value a bone loss is diagnosed. In this example, the one or more periodontal structures include both determined bone level and bone loss. The threshold value may be between 3 and 5 mm, 5 and 8 mm, 3 and 10 mm or above 10 mm.

The previous described example explains the determination of the periodontal structures and generation of the combined data for the training dataset with the use of segmentation. However, as previously mentioned and discussed, the segmentation of the extra-oral image data may not be needed as long as mapping matrix comprising tooth information and correlated determined periodontal structures is obtained. The given examples have been provides for ease of illustration but should not be considered as the only possible ways of implementing the invention as described herein.

In both examples illustrated in FIGS. 3A and 3B, the combining 100D of the extra-oral image data 10 and the intra-oral scan data 20 is using the segmented teeth (2A, 2B) of the extra-oral image data 10 and the segmented teeth (5A, 5B) of the intra-oral scan data 20 as reference surfaces. Alternatively, the combining 100D of the extra-oral image data 10 and the intra-oral scan data 20 is based on an alignment of the intra-oral scan data 20 with the extra-oral image data 10 using the segmented teeth (2A, 2B) of the extra-oral image data 10 and the segmented teeth (5A, 5B) of the intra-oral scan data 20 as reference surfaces. Alternatively, the segmentation process may be left out and the training data set comprises a mapping matrix including information about teeth and periodontal structures identified for the respective teeth. That is, the mapping matrix could comprise tooth numbering and corresponding periodontal structured determined for the respective teeth in the mapping matrix.

FIG. 3C illustrates an example of the method 200 outputting diagnostic data 60 of a patient based on the training data set 30. In this example, the method 200 receives 200A intra-oral scan data 50 of a patient, and which is segmented into segmented teeth (6A, 6B) and segmented soft tissue 7, and in this example, the segmented soft tissue 7 includes a soft tissue edge 43. The segmented teeth 6A are then aligned with segmented teeth (5A, 5B) of the training data set 30. In this example, the training data set 30 includes segmented teeth (5A, 5B) and segmented bone 3. The segmented teeth (6A, 6B) of the intra-oral scan data 50 are then aligned 200B with the segmented teeth (5A, 5B) of the training data set 30, and then at least the segmented bone 3 is retrieved from the training data set 30 which corresponds to the segmented teeth (5A, 5B) of the training data set 30.

Alignment of training data set 30 with intra-oral scan data 50 includes finding the best or most optimal correlation of the segmented teeth (5A, 5B) of the training data set and the segmented teeth (6A, 6B) of the intra-oral scan data 50 of the patient.

The segmented bone 3 of the training data set 30 is then combined with the segmented teeth (6A, 6B) and the segmented soft-tissue 7. In this example, the diagnostic data 60 includes the combined data and periodontal structures, and the periodontal structures includes the bone level determined by the distances (33A, 33B, 33C) between reference sites (31A, 31B, 31C) arranged on the tooth 6A and reference sites arranged on the bone edge 41. In another example, the diagnostic data includes only the periodontal structures which in this example is the bone level. FIG. 3D illustrates a similar example as in FIG. 3C, but in FIG. 3D the distances (33A, 33B, 33C) are determined between a cementoenamel junction 40 and a bone edge 41.

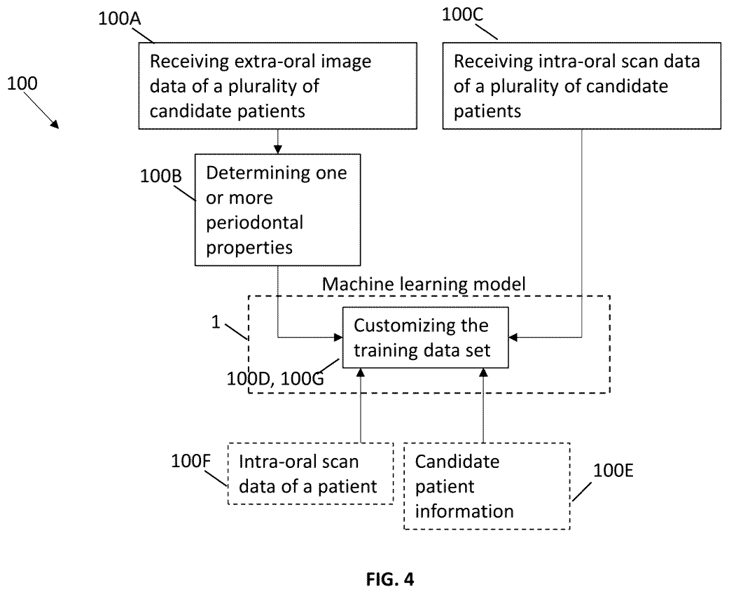

FIG. 4 illustrates an example of the method 100 where the training data set is being trained for a specific patient. In this example, the training data set is being customized for the specific patient. In this example the machine learning model is still being trained 100D by the extra-oral image data of the plurality of candidate patients and the corresponding intra-oral scan data, and alternatively, by patient information. Furthermore, the training data set of the machine learning model 1 is being customized for a patient by receiving 100F intra-oral scan data of the patient.

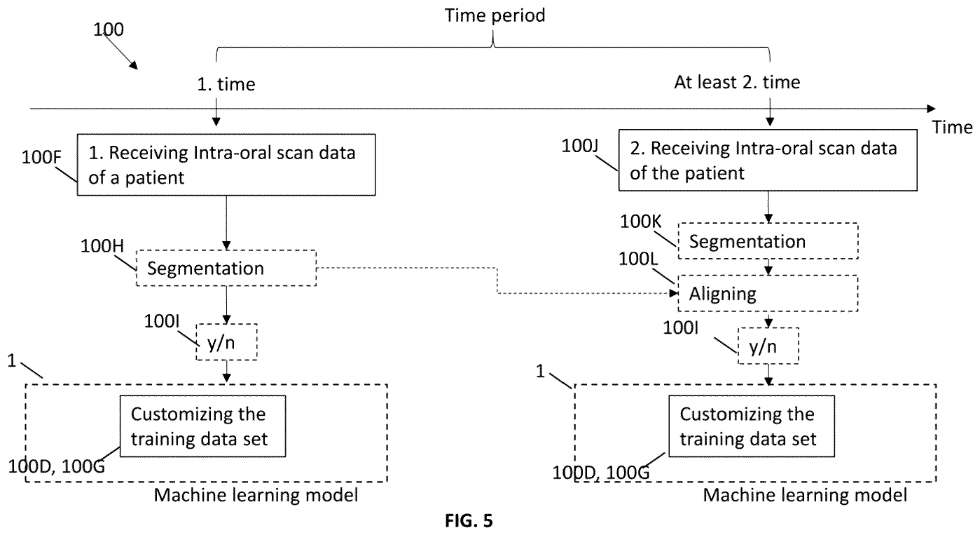

FIG. 5 illustrates an example of the method 100 which generates 100D training data set and customizing 100G the training data set to a specific patient. In this example, the method includes receiving 100F intra-oral scan data of the patient at a first time and within a time period. A segmentation 100H of the intra-oral scan data is performed for customizing 100G the training data set. The method 100 further includes receiving 100J intra-oral scan data of the patient at a second time and within the time-period. The intra-oral scan data received at the second time is segmented 100K, and the segmented intra-oral scan data received at the second time is aligned with the previous segmented intra-oral scan data received at the first time. The training data set is then customized 100G based on a difference in the alignment 100L. Alternatively, the customizing of the training data set is only allowed 100I based on one or more of the following criteria: quality of the intra-oral scan data of the patient, and level of mismatch between intra-oral scan data of the patient and the training data set.

As previously described, the customization may be configured as an update of the already once trained machine learning model to include information gathered from a patient scan at a clinical visit and reference is made to these sections.

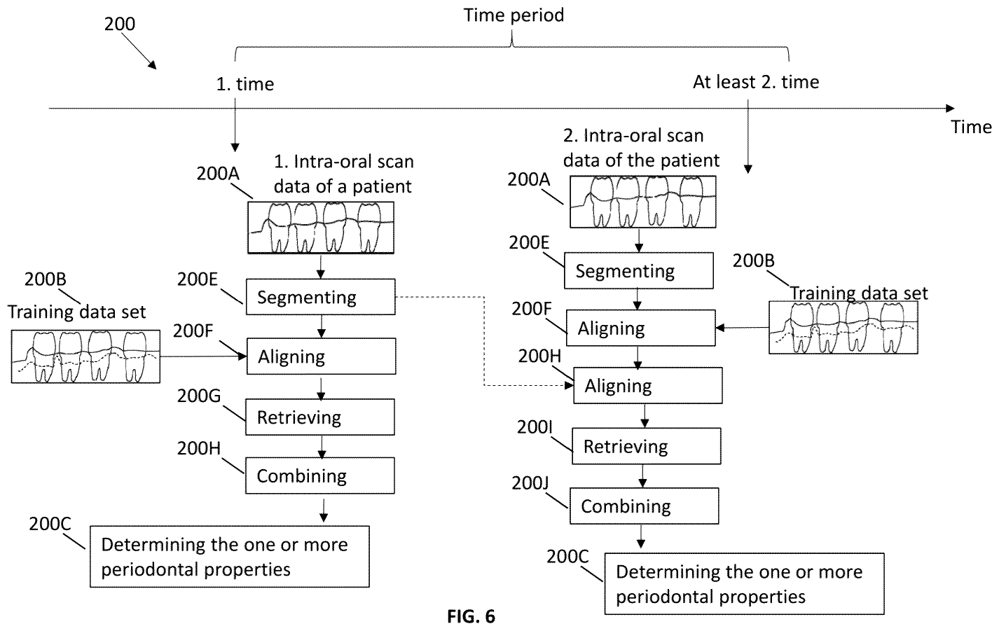

FIG. 6 illustrates an example of the method 200 which includes receiving 200A intra-oral scan data of the patient at a first time and within a time-period, segmenting 200E the intra oral scan data of the patient into teeth and soft tissue based on an intra-oral segmentation algorithm, aligning 200F the segmented teeth of the intra-oral scan data of the patient with segmented teeth of the training data set, retrieving 200G from the training data set at least the segmented bone corresponding to the segmented teeth of the training data set, combining 200H the segmented bone with the segmented teeth of the intra-oral scan data of the patient, and determining 200C the one or more periodontal structures based on the retrieved segmented bone and the segmented teeth of the intra-oral scan data of the patient. Then, the patient is being scanned once again with an intra-oral scanner. The method 200 includes receiving 200A intra-oral scan data of the patient at a second time and within the time-period, segmenting 200E the intra oral scan data of the patient received at the second time into teeth and soft tissue based on the intra-oral segmentation algorithm, aligning 200F the segmented teeth of the intra-oral scan data received at the second time with segmented teeth of the training data set, determining 200H a difference in an alignment of the segmented teeth and soft tissue of the intra-oral scan data received at the first time and at the second time, retrieving 200I from the training data at least the segmented bone corresponding to the determined difference in the alignment, combining 200J the segmented bone with the segmented teeth of the intra-oral scan data received at the second time, and determining 200C the one or more periodontal structures based on the retrieved segmented bone and the segmented teeth of the intra-oral scan data of the patient.

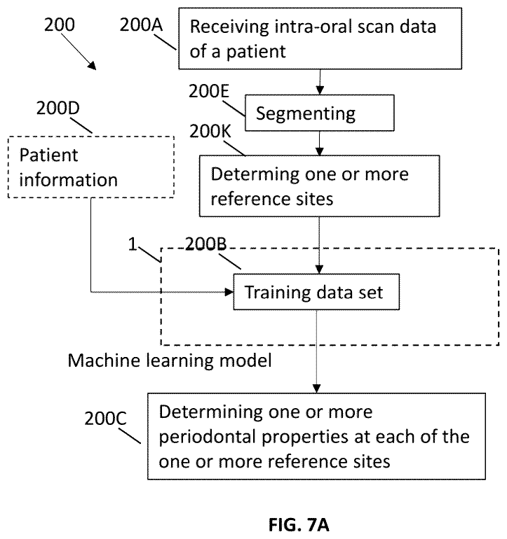

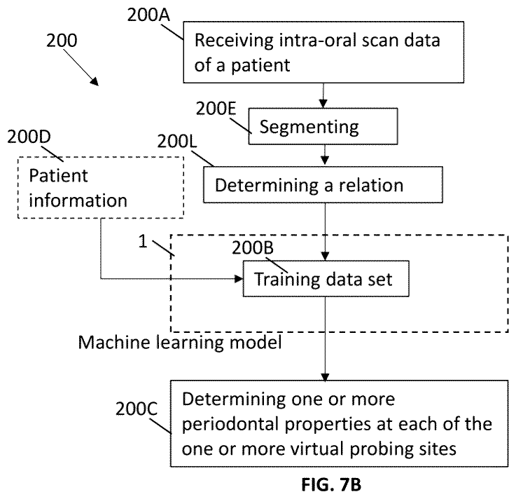

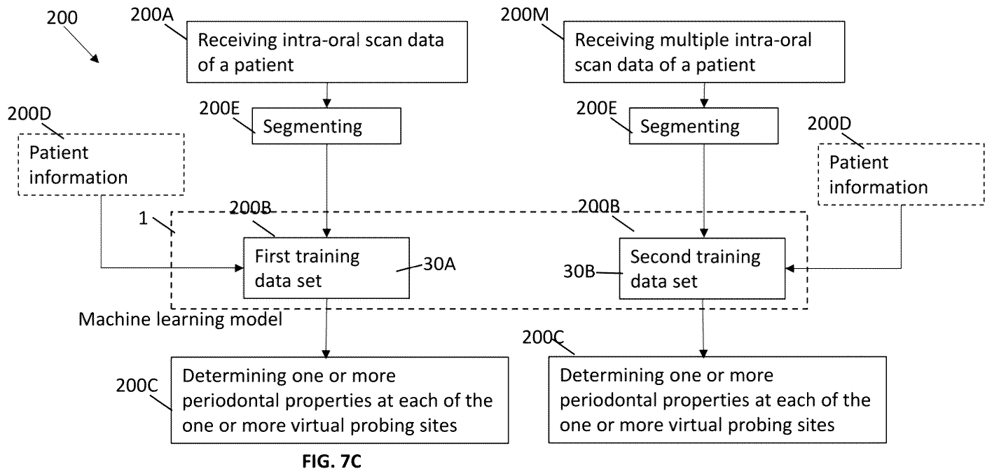

FIG. 7A illustrates the method 200 of using the trained machine learning model to output a diagnostic dataset. The method illustrated in this example includes further determining 200K one or more reference sites per identified tooth of the segmented teeth of the intra-oral scan data of the patient, and determining 200C the one or more periodontal structures of the patient by correlating the intra-oral scan data of the patient with the training data set at each of the one or more reference sites.

FIG. 7B illustrates the method 200 of using the trained machine learning model to output a diagnostic dataset. The method illustrated in this example includes further determining 200L a relation between the segmented teeth and soft-tissue of the patient, correlating 200C the relation with the training data which includes a correlation of a relation between segmented teeth and segmented bone provided by the extra-oral image data of the plurality of candidate patients and a relation between segmented teeth and segmented soft tissue provided by the intra-oral scan data of the plurality of candidate patients.

FIG. 7C illustrates the method 200 as described in any of the previous figures. Thus, FIG. 7C the machine learning model 1 includes a first training data set 30A configured to receive 200A a single intra-oral scan of the patient and a second training data set 30B configured to receive 200M multiple scan for each of the plurality of the patient. For example, if multiple intra-oral scans are being performed on the same patient, the machine learning model 1 is then configured to use the second training data set 30B for providing diagnostic data set.

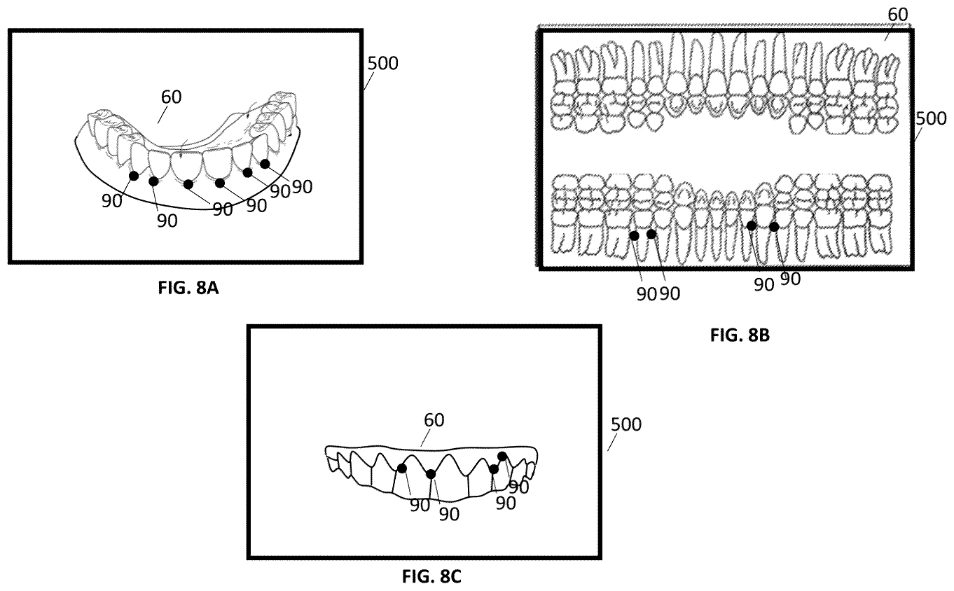

FIGS. 8A-8C illustrates an example where the one or more periodontal structures 90 as output from the trained machine learning model are presented on a graphical user interface 500. The one or more periodontal structures may be visualized with color or text. In FIG. 8A the periodontal structures 90 are visualized on a 3D model of a mouth anatomy determined by the diagnostic data 60 which includes the segmented teeth and soft tissue of the intra-oral scan data 50 of the patient combined with the segmented bone retrieved from the training data set 30. FIG. 8C illustrates a 2D model of the mouth anatomy determined by the diagnostic data 60.

In an example, the intra-oral scan data and the diagnostic data set as output from the machine learning model can be post-processed into various forms to be displayed in e.g. the graphical user interface 500. By using e.g. back-propagation to the output from the machine learning model it is possible to visualize, as illustrated in FIG. 8A, the output from the machine learning model on the previous unseen image input. That is, the previous unseen image in the form of an intra-oral scan data which has not previously been seen by the machine learning model may be visualized in the graphical user interface 500. In FIG. 8A illustrated as intra-oral scan data 60 of a lower jaw and in FIG. 8C intra-oral scan data of an upper jaw. As can been seen in FIGS. 8A and 8C, the periodontal structures 90 as output from the machine learning model in the form of a diagnostic data set may be visualized on the locations of the tooth at which the periodontal structures were determined by the machine learning model. Thus, it is possible to visualize the input image of e.g. an upper or lower jaw and fuse onto the image by e.g. image warping techniques the output from the machine learning model to efficiently illustrate to a user the findings of periodontal structures at the specific input image.

Accordingly, by using the trained neural network (as described herein) on new image data it is possible to assess without the use of radiation if the patient suffers from periodontal disease. In dependence of the neural network used and the input data that the neural network has been trained on, the assessment of periodontal disease may include identification of the location on a tooth where a bone loss is present, the amount of bone level for a specific tooth, the age of the person, the gender of the person, potential other disease related to specific teeth of the input image data as acquired from an intra-oral scanner.

FIG. 8B illustrates a dental chart including periodontal structures marked at the reference sites as output from the diagnostic data 60. This illustrates the possibility of transferring the output from the machine learning model to a periodontal charging system in e.g. a practice management system to ensure that patients journals are updated with the most recent medical information.

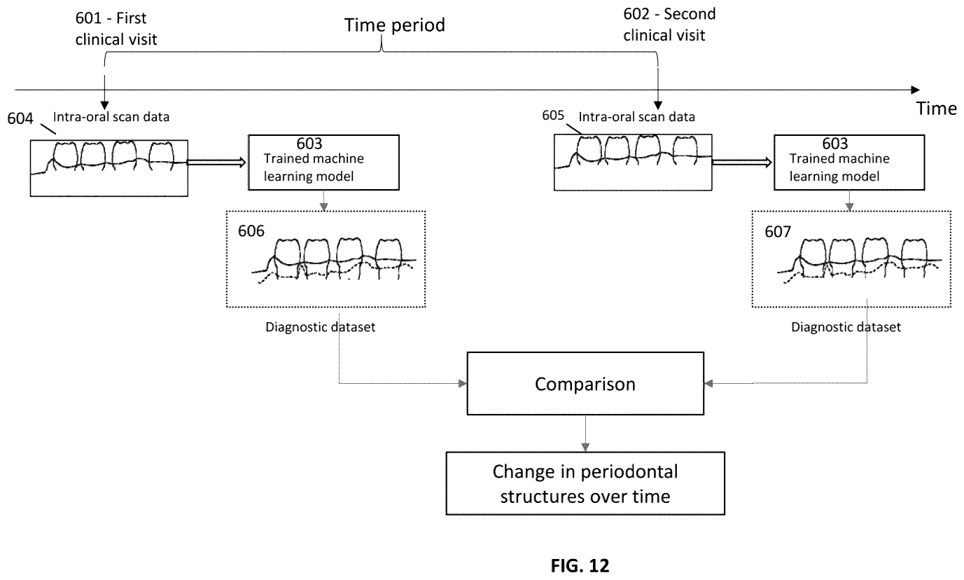

As previously described in relation to FIG. 11, the trained machine learning model is configured to output a diagnostic data set comprising a prediction of the presence and potential also the location of determined periodontal structures in the input intra-oral scan data. Accordingly, with reference now to FIG. 12, illustrating a first clinical visit 601 to the left, when a patient is being scanned by an intra-oral scanner, the method described herein allows using a trained machine learning model 603 to identify if periodontal structures are present in the intra-oral scan 604 obtained from the patient. If periodontal structures are identified by the machine learning model 603 these are output as part of the diagnostics dataset 606. As can be seen from the illustrated diagnostic data set 606, the intra-oral scan data 604 as input to the machine learning model 603 have been found by the machine learning model to contain periodontal structures as illustrated by the dotted line in 606 of FIG. 12.

It is noted that the periodontal structures could be any of the ones mentioned in relation to FIGS. 3A and 3B, where specifically bone edge, CEJ and reference sites are mentioned. The illustrating given in FIG. 12 is only presented for illustrative purpose for easing the understanding of the disclosure. Accordingly, it should be understood that diagnostic data as output from the machine learning model could be any of a segmentation map, object recognition map, label classification etc.

The diagnostic dataset 606 provides the dental practitioner with a first assessment of periodontal diseases of a patient at a first point in time without using ionizing radiation to evaluate the periodontal structures of the patient. The diagnostic data obtained at the first visit 601 may be stored in a practice management system in connection with a patient history file. When the patient visits the dental practitioner for a follow up visit at a second clinical visit 602, the dental practitioner may acquire a new second visit scan 605 of the patient. As illustrated in FIG. 12, the new second visit scan 605 is configured to be input to the same machine learning model 603 as previously run on the first visit scan data 604. The machine learning model 603 is configured to process the second visit scan data 605 to output a diagnostic data set providing a prediction of the current state of the periodontal structures of the second visit scan data. By comparing the two diagnostic dataset 606, 607 obtained at two different times (i.e. at first and second visit) the methods described herein comprises inputting the diagnostic dataset alone or tougher with the intra-oral scan data to a comparison processor, which is configured to compare the first and second diagnostic dataset 606, 607 to assess and output a change in periodontal structure over time. In this way it is possible to assess if potential periodontal diseases identified at the first clinical visit have progressed from the first clinical visit to the second clinical visit, without using ionizing radiation.

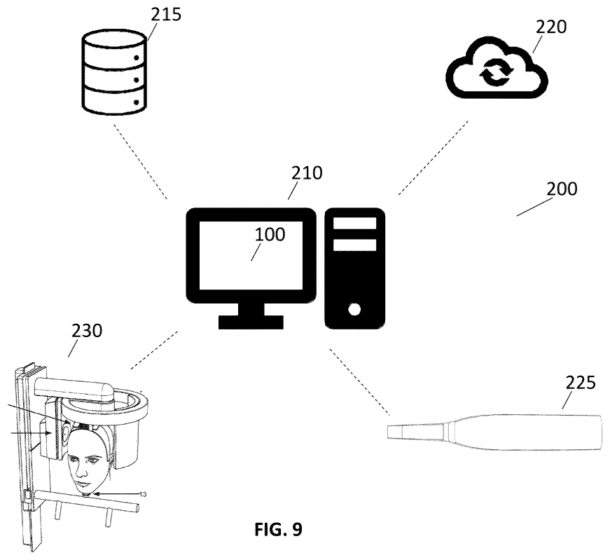

Turning now to FIG. 9, which illustrates a dental scanning system 200 which includes means for carrying out the methods (100, 200). The system includes a computer 210 which has a wired or wireless interface to a server 215, a cloud server 220, an intra-oral scanner 225 and/or an extra-oral image device 230. The extra-oral image data of the plurality of candidate patients and/or the intra-oral scan data of the plurality of candidate patients may be stored on the server 215, a memory of the computer 210, or a cloud server 220. The training data set may be stored on the server 215, the memory of the computer 210, or the cloud server 220.

The computer 210 may include a data processing device configured to carry out the methods (100, 200).

The computer 210 may include a computer-readable storage medium configured to cause the computer 210 to carry out the methods (100, 200).

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

### List of Items

Shayeb K N A A, Turner W, Gillam D G. “Periodontal Probing: A Review”. Primary Dental Journal. 2014; 3 (3): 25-29. doi: 10.1308/205016814812736619;

Dimitrios Kloukos, DMD, Dr Med Dent/George Koukos, DMD/Nikolaos Gkantidis, Priv-Doz Dr Med Dent/Andreas Stavropoulos, Prof, DDS, PhD, Dr odont. “Transgingival probing: a clinical gold standard for assessing gingival thickness” Quintessence international, Volume 52, number 5, May 2021.

Item 1. A computer-implemented method of generating a training data set for a machine learning model, wherein the method comprising;
receiving extra-oral image data of a plurality of candidate patients provided by an extra-oral image device;
determining one or more periodontal structures for each of the plurality of candidate patients based on the received extra-oral image data;
receiving intra-oral scan data of the plurality of candidate patients provided by an intra-oral scanner, and
generating a training data set by combining the extra-oral image data with the intra-oral scan data for each of the plurality of candidate patients for aligning the one or more periodontal structures to the intra-oral scan data.

Item 2. A computer implemented method according to item 1, further comprising receiving candidate patient information of the plurality of candidate patients and generating the training data set by combining the received patient information for each of the plurality of candidate patients with the combined extra-oral image data and the intra-oral scan data for each of the plurality of candidate patients.

Item 3. A computer implemented method according to any of the preceding items, wherein the one or more periodontal structures include bone loss or bone level.

Item 4. A computer implemented method according to any of the preceding items, further comprising segmenting the extra-oral image data of the plurality of candidate patients into teeth and bone based on an extra-oral segmentation algorithm, and segmenting the intra-oral scan data of the plurality of candidate patients into teeth and soft tissue based on an intra-oral segmentation algorithm.

Item 5. A computer implemented method according to item 4, wherein the combining of the extra-oral image data and the intra-oral scan data is using the segmented teeth of the extra-oral image data and the segmented teeth of the intra-oral scan data as reference surfaces.

Item 6. A computer implemented method according to item 4, wherein the combining of the extra-oral image data and the intra-oral scan data is based on an alignment of the intra-oral scan data with the extra-oral image data using the segmented teeth of the extra-oral image data and of the intra-oral scan data as reference surfaces.

Item 7. A computer implemented method according to any of items 4 to 6, wherein the determining of the one or more periodontal structures includes a bone level which is determined for each of the segmented teeth in the extra-oral image data by a distance between a bone edge determined by the segmented bone and a cementoenamel junction, and where the cementoenamel junction is determined by the segmented teeth of the extra-oral image data.

Item 8. A computer implemented method according to any of item 4 to 7, wherein the determining of the one or more periodontal structures for each of the plurality of candidate patients includes determining a first relation between the segmented teeth and bone of the extra-oral image data, and where the combining of the extra-oral image data with the intra-oral scan data includes aligning the first relation with the intra-oral scan data.

Item 9. A computer implemented method according to item 7, wherein the combining of the extra-oral image data with the intra-oral scan data includes an alignment of the determined distance for the extra-oral image data with the intra-oral scan data.

item 10. A computer implemented method according to item 4, wherein the determining of the one or more periodontal structures for each of the plurality of candidate patients includes:
determining one or more reference sites per identified tooth of the segmented teeth of the extra-oral image data, and
determining the one or more periodontal structures at each of the one or more reference sites.

Item 12. A computer implemented method according to any of the preceding items:
receiving intra-oral scan data of a patient at a first time and within a time-period, and
customizing the training data set to the patient by including the intra-oral scan data of the patient.

Item 13. A computer implemented method according to any of item 1 to 11:
receiving intra-oral scan data of a patient at a first time and within a time-period,
receiving intra-oral scan data of the patient at a second time and within the time-period,
aligning the intra-oral scan data received at the first time and at the second time, and
customizing the training data set to the patient based on a difference in the alignment of the intra-oral scan data received at the first time and at the second time.

Item 14. A computer implement method according to any of items 12 and 13, further comprising determining whether to customize the training data set to the patient based on one or more of following criteria:
quality of the intra-oral scan data of the patient, and
level of mismatch between intra-oral scan data of the patient and the training data set.

### Operating the Computer Implemented Method for Providing a Diagnostic Data Set

Item 15. A computer-implemented method for providing a diagnostic data set of a patient based on the computer-implemented method of generating a training data set according to any of item 1 to 14, comprising receiving intra-oral scan data of the patient, and outputting a diagnostic data set of the patient, where the diagnostic data set is determined by processing the intra-oral scan data of the patient based on the training data set.

Item 16. A computer implemented method according to item 15, comprising receiving patient information of the patient, and where the diagnostic data set is determined by processing the intra-oral scan data of the patient and patient information of the patient based on the training data set.

Item 17. A computer implemented method according to any of items 15 and 16, wherein the outputting of the diagnostic data set of the patient includes one or more periodontal structures determined by the processing of the intra-oral scan data of the patient based on the training data set.

Item 18. A computer implemented method according to any of items 15 and 16, wherein the outputting of the diagnostic data set of the patient includes the one or more periodontal structures determined by:
receiving intra-oral scan data of the patient at a first time and within a time-period,
segmenting the intra oral scan data received at the first time into teeth and soft tissue based on an intra-oral segmentation algorithm,
aligning the segmented teeth of the intra-oral scan data received at the first time with segmented teeth of the training data set,
retrieving from the training data set at least the segmented bone corresponding to the segmented teeth of the training data set,
combining the segmented bone with the segmented teeth of the intra-oral scan data received at the first time, and
determining the one or more periodontal structures based on the retrieved segmented bone and the segmented teeth of the intra-oral scan data of the patient.

Item 19. A computer implemented method according to item 18, wherein the outputting of the diagnostic data set of the patient includes the one or more periodontal structures determined by:
receiving intra-oral scan data of the patient at a second time and within the time-period,
segmenting the intra oral scan data of the patient received at the second time into teeth and soft tissue based on the intra-oral segmentation algorithm,
aligning the segmented teeth of the intra-oral scan data received at the second time with segmented teeth of the training data set,
determining a difference in an alignment of the segmented teeth and soft tissue of the intra-oral scan data received at the first time and at the second time,
retrieving from the training data at least the segmented bone corresponding to the determined difference in the alignment,
combining the segmented bone with the segmented teeth of the intra-oral scan data received at the second time, and
determining the one or more periodontal structures based on the retrieved segmented bone and the segmented teeth of the intra-oral scan data of the patient.

Item 20. A computer implemented method according to any of items 18 and 19, wherein the determining of the one or more periodontal structures of the patient includes:
determining one or more reference sites per identified tooth of the segmented teeth of the intra oral scan data received at the first time or at the second time, and
determining the one or more periodontal structures of the patient by correlating the intra-oral scan data received at the first time or at the second time with the training data set at each of the one or more reference sites.

Item 21. A computer implemented method according to item 20, wherein the determining of the one or more periodontal structures at each of the one or more reference sites includes;
determining a relation between the segmented teeth and soft-tissue of the patient,
correlating the relation with the training data which includes a correlation of a relation between the segmented teeth and the segmented bone provided by the extra-oral image data of the plurality of candidate patients and a relation between the segmented teeth and the segmented soft tissue provided by the intra-oral scan data of the plurality of candidate patients.

Item 22. A computer implemented method according to any of items 15 to 21, wherein the one or more periodontal structures includes bone loss or bone level.

Item 23. A computer implemented method according to any of items 15 to 22, wherein the one or more periodontal structures are presented on a graphical user interface including a 2D model or a 3D model of a mouth anatomy determined by the diagnostic data on which the one or more periodontal structures are visualized.

Item 24. A computer implemented method according to items 20 and 23, wherein the one or more periodontal structures are visualized at the one or more reference sites.

Item 25. A computer implemented method according to any of items 23 and 24, wherein the one or more periodontal structures are visualized with color or text.

Item 26. A computer implemented method according to any of items 23 to 25, wherein the one or more periodontal structures are visualized if fulfilling a visualization criterium.

Item 27. A computer implemented method according to item 22 and 26, wherein the visualization criterium defines that the bone level is above a threshold level.

Item 28. A computer implemented method according to item 27, wherein the threshold level is between 2 mm and 3 mm, around 3 mm, or above 3 mm.

### Device and System Items

Item 29. A computer program comprising instructions which, when the program is executed by a computer, cause the computer to carry out the method of any items 1 to 28.

Item 30. A dental scanning system comprising means for carrying out the method of any items 1 to 28.

Item 31. A dental scanning system according to item 30, wherein the extra-oral image data of the plurality of candidate patients and/or the intra-oral scan data of the plurality of candidate patients are stored on a server, a memory of the computer, or a cloud server part of the dental scanning system.

Item 32. A dental scanning system according to any of items 30 and 31, wherein the training data set are stored on the server, the memory of the computer, or the cloud server.

Item 33. A data processing device comprising means for carrying out the method of any items 1 to 28.

Item 34. A computer-readable storage medium comprising instructions which, when executed by a computer, cause the computer to carry out the method of any item 1 to 28.

1. A computer-implemented method of generating a training data set for a machine learning model, the method comprising:
receiving extra-oral image data of a plurality of candidate patients provided by an extra-oral image device;
determining one or more periodontal structures for each of the plurality of candidate patients based on the received extra-oral image data;
receiving intra-oral scan data of the plurality of candidate patients provided by a handheld intra-oral scanner, and
generating a training data set by combining determined extra-oral one or more periodontal structures with the intra-oral scan data for each of the plurality of candidate patients for aligning the one or more periodontal structures to the intra-oral scan data.

2. A computer implemented method according to claim 1, wherein the combining comprises
identifying corresponding teeth in the extra-oral image data and the intra-oral scan data; and
determining in the extra-oral image data one or more periodontal structures; and
using an image alignment process to map or correlate the determined periodontal structures with corresponding teeth in the intra-oral scan data.

3. A computer implemented method according to claim 1, wherein the determining of the one or more periodontal structures for each of the plurality of candidate patients comprises
identifying in the extra-oral image data a plurality of teeth;
determining one or more reference sites per identified tooth of the extra-oral image data; and
determining the one or more periodontal structures at each of the one or more reference sites.

4. A computer implemented method according to claim 2, wherein the method comprises determining for each reference site a tooth reference point and a bone reference point using the extra-oral image data.

5. A computer implemented method according to claim 3, comprising
determining a first relation between teeth and bone in the extra-oral image data using the one or more reference sites per identified tooth; and
combining the determined first relation from the extra-oral image data with the intra-oral scan data by aligning the first relation with the intra-oral scan data.

6. A computer implemented method according to claim 5, wherein the first relation comprises a distance between the tooth reference point arranged on a tooth of the extra-oral image data and a bone reference point arranged on a bone which is closest to the tooth reference point or at least in vicinity to the tooth reference point.

7. A computer implemented method according to claim 4, wherein the determining of the one or more periodontal structures includes determining a bone level for each of the teeth in the extra-oral image data, wherein the bone level is determined by a distance between an identified bone reference point and an identified cementoenamel junction of the extra-oral image data, and wherein the determined bone level for the extra-oral image data is merged with the intra-oral scan data.

8. A computer implemented method according to claim 1, further comprising
segmenting the extra-oral image data of the plurality of candidate patients into teeth and bone based on an extra-oral segmentation algorithm, and
segmenting the intra-oral scan data of the plurality of candidate patients into teeth and soft tissue based on an intra-oral segmentation algorithm.

9. A computer implemented method according to claim 8, wherein the combining of the extra-oral image data and the intra-oral scan data is using the segmented teeth of the extra-oral image data and the segmented teeth of the intra-oral scan data as reference surfaces.

10. A computer implemented method according to claim 9, wherein the combining of the extra-oral image data and the intra-oral scan data is based on an alignment of the intra-oral scan data with the extra-oral image data using the segmented teeth of the extra-oral image data and of the intra-oral scan data as reference surfaces.

11. A computer-implemented method for providing a diagnostic data set of a patient comprising
receiving the intra-oral scan data of the patient;
inputting to the trained machine learning model according to claim 1, the intra-oral scan data; and
receiving from the trained machine learning model a diagnostic dataset of the patient.

12. A computer implemented method according to claim 11, wherein the outputting of the diagnostic data set of the patient includes one or more periodontal structures determined by the processing of the intra-oral scan data of the patient based on the training data set.

13. A computer implemented method according to claim 11, wherein the diagnostic data comprises a prediction that a periodontal structure is present on one or more teeth in the intra-oral scan data and at what location that prediction is present.

14. A computer implemented method according to claim 1, wherein the one ore more periodontal structures are presented on a graphical user interface including a 2D model or a 3D model of a mouth anatomy determined by the diagnostic data on which the one or more periodontal structures are visualized.

15. A computer implemented method according to claim 14, wherein the one or more periodontal structures are visualized at the one or more reference sites when a value of the one or more periodontal structures fulfills a visualization criterium.
